(19) United States
(12) Patent Application Publication
(10) Pub. No.: US 2011/0318417 A1
(43) Pub. Date: Dec. 29, 2011

(54) HIGHLY PURE CINACALCET OR A PHARMACEUTICALLY ACCEPTABLE SALT THEREOF

(75) Inventors: Sonny Sebastian, Kerala (IN); Seetha Rama Sarma Peri, Andhra Pradesh (IN); Katikireddy Ramamurthy, Andhra Pradesh (IN); Nitin Sharadchandra Pradhan, Maharashtra (IN)

(73) Assignee: ACTAVIS GROUP PTC EHF, Hafnarfjordur (IS)

(21) Appl. No.: 13/133,276

(22) PCT Filed: Dec. 8, 2009
(86) PCT No.: PCT/IB09/07932

§ 371 Date: Aug. 8, 2011

(30) Foreign Application Priority Data
Dec. 8, 2008 (IN) 3086/CHE/2008
Feb. 10, 2009 (IN) 282/CHE/2009

(51) Int. Cl. A61K 9/14; A61K 31/137; A61P 5/18; C07C 211/30

(52) U.S. Cl. 424/489; 564/387; 564/374; 514/654; 514/655

(57) ABSTRACT

Provided herein are impurities of cinacalcet, (R)-α-methyl-N-[3-[3-(trifluoromethyl)phenyl]propyl]-1-(5,6,7,8-tetrahydronaphthalene)methaneamine (tetrahydro cinacalcet impurity), (R)-α-Methyl-N-[3-[3-(trifluoromethyl)phenyl]propyl]-1-naphthalenemethaneamine-N-oxide (cinacalcet N-oxide impurity) and (R)-α-methyl-N-[3-[3-(trifluoromethyl)phenyl]methyl]-1-naphthalenemethaneamine (benzylamine impurity); and processes for preparation and isolation thereof. Provided further herein is a highly pure cinacalcet or a pharmaceutically acceptable salt thereof substantially free of impurities, processes for the preparation thereof, and pharmaceutical compositions comprising highly pure cinacalcet or a pharmaceutically acceptable salt thereof substantially free of impurities.

### CROSS REFERENCE TO RELATED APPLICATION

This application claims the benefit of priority to Indian provisional application Nos. 3086/CHE/2008, filed on Dec. 8, 2008; and 282/CHE/2009, filed on Feb. 10, 2009; which are incorporated herein by reference in their entirety.

### FIELD OF THE DISCLOSURE

Disclosed herein are impurities of cinacalcet or a pharmaceutically acceptable salt thereof, and processes for the preparation and isolation thereof. Disclosed further herein is a highly pure cinacalcet or a pharmaceutically acceptable salt thereof substantially free of impurities, processes for the preparation thereof, and pharmaceutical compositions comprising highly pure cinacalcet or a pharmaceutically acceptable salt thereof substantially free of impurities.

### BACKGROUND

Cinacalcet, chemically known as (R)-α-methyl-N-[3-[3-(trifluoromethyl)phenyl]propyl]-1-naphthalenemethane amine, is an important antihyperparathyroic agent that acts as a calcimimetic by allostric activation of the calcium sensing receptor that is expressed in various human organ tissues. Cinacalcet is used to treat secondary hyperparathyroidism in patients with chronic kidney disease and hypercalcemia in patients with parathyroid carcinoma. Cinacalcet hydrochloride is sold by Amgen under the trade name SENSIPAR™ in the USA and as MIMPARA™ in Europe. Cinacalcet hydrochloride is represented by the following structural formula I:

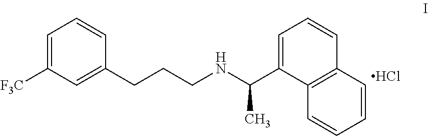

U.S. Pat. No. 6,011,068 generally describes cinacalcet and its pharmaceutically acceptable acid addition salts.

U.S. Pat. No. 6,211,244 describes cinacalcet and related compounds, and their pharmaceutically acceptable salts.

Processes for the preparation of cinacalcet and related compounds, and their pharmaceutically acceptable salts are disclosed in U.S. Pat. Nos. 6,211,244; 7,250,533; 5,648,541; 7,247,751; and 7,393,967; PCT Publication Nos. WO06/127933; WO06/125026; WO06/127941; WO07/062147; WO07/112280; WO07/127445; WO07/127449; WO08/058235; WO08/000423; WO08/035212; WO08/058236; WO08/063645; and WO08/068625.

According to U.S. Pat. No. 6,211,244, cinacalcet or its analogues are prepared by the reaction of 3-[(3-trifluoromethyl)phenyl]cinnamaldehyde or a derivative thereof with R-(+)-1-(1-naphthyl)ethyl amine or a derivative thereof in the presence of titanium(IV)isopropoxide. The resulting intermediate imines are reduced in situ by the action of sodiumcyanoborohydride, sodiumborohydride or sodium triacetoxyborohydride. The intermediate enamine is catalytically reduced using palladium or palladium hydroxide on carbon to produce cinacalcet base or its analogues. Hydrochlorides of these analogues are prepared by the precipitation using gaseous HCl in ether or hexane in combination with gaseous HCl in ether.

U.S. Pat. No. 7,294,735 discloses an impurity of cinacalcet, cinacalcet carbamate, and process for the preparation thereof. The patent also discloses a cinacalcet salt having cinacalcet carbamate in an amount of about 0.03 area percent to about 0.15 area percent, and a process for the preparation thereof.

Cinacalcet obtained by the processes described in the above prior art does not have satisfactory purity for pharmaceutical use. Unacceptable amounts of impurities are generally formed along with cinacalcet. In addition, the processes involve the additional step of column chromatographic purifications. Methods involving column chromatographic purifications are generally undesirable for large-scale operations as they require additional expensive setup adding to the cost of production, thereby making the processes commercially unfeasible.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in cinacalcet or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for highly pure cinacalcet or a pharmaceutically acceptable salt thereof substantially free of impurities, as well as processes for preparing thereof.

### SUMMARY

In one aspect, provided herein is a tetrahydro cinacalcet compound, (R)-α-methyl-N-[3-[3-(trifluoromethyl)phenyl]propyl]-1-(5,6,7,8-tetrahydronaphthalene)methane amine, having the following structural formula A:

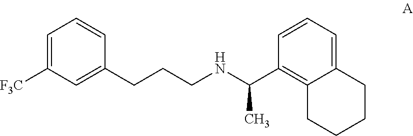

or a pharmaceutically acceptable acid addition salt thereof.

In another aspect, provided herein is an impurity of cinacalcet, tetrahydro cinacalcet, (R)-α-methyl-N-[3-[3-(trifluoromethyl)phenyl]propyl]-1-(5,6,7,8-tetrahydronaphthalene)methane amine, of formula A.

In another aspect, encompassed herein is a process for synthesizing and isolating the tetrahydro cinacalcet of formula A, also referred to as the “tetrahydro cinacalcet impurity”.

In another aspect, provided herein is a cinacalcet N-oxide compound, (R)-α-Methyl-N-[3-[3-(trifluoromethyl)phenyl]propyl]-1-naphthalenemethaneamine-N-oxide, having the following structural formula B:

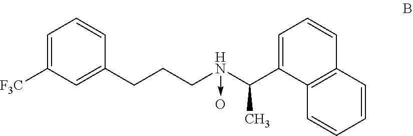

In another aspect, provided herein is an impurity of cinacalcet, cinacalcet N-oxide impurity, (R)-α-Methyl-N-[3-[3-(trifluoromethyl)phenyl]propyl]-1-naphthalenemethaneamine-N-oxide, of formula B.

In another aspect, encompassed herein is a process for synthesizing and isolating the cinacalcet N-oxide compound of formula B, also referred to as the “cinacalcet N-oxide impurity”.

In another aspect, provided herein is a benzylamine compound, (R)-α-methyl-N-[3-[3-(trifluoromethyl)phenyl]methyl]-1-naphthalenemethaneamine, having the following structural formula C:

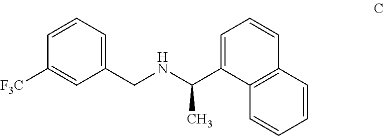

In another aspect, provided herein is an impurity of cinacalcet, benzylamine impurity, (R)-α-methyl-N-[3-[3-(trifluoromethyl)phenyl]methyl]-1-naphthalenemethaneamine, of formula C.

In another aspect, encompassed herein is a process for synthesizing and isolating the cinacalcet benzylamine compound of formula C, also referred to as the “cinacalcet benzylamine impurity”.

In another aspect, provided herein is a highly pure cinacalcet or a pharmaceutically acceptable salt thereof substantially free of tetrahydro cinacalcet impurity.

In another aspect, provided herein is a highly pure cinacalcet or a pharmaceutically acceptable salt thereof substantially free of at least one, or more, specifically all, of the tetrahydro cinacalcet impurity, cinacalcet N-oxide impurity, cinacalcet benzylamine impurity, and ‘0.66 RRt’ impurity.

In another aspect, encompassed herein is a process for preparing the highly pure cinacalcet or a pharmaceutically acceptable salt thereof substantially free of tetrahydro cinacalcet impurity.

In yet another aspect, encompassed herein is a process for preparing the highly pure cinacalcet or a pharmaceutically acceptable salt thereof substantially free of at least one, or more, specifically all, of the tetrahydro cinacalcet impurity, cinacalcet N-oxide impurity, cinacalcet benzylamine impurity, and ‘0.66 RRt’ impurity.

In another aspect, provided herein is a pharmaceutical composition comprising highly pure cinacalcet or a pharmaceutically acceptable salt thereof substantially free of at least one, or more, specifically all, of the tetrahydro cinacalcet impurity, cinacalcet N-oxide impurity, cinacalcet benzylamine impurity, and ‘0.66 RRt’ impurity, and one or more pharmaceutically acceptable excipients.

In still another aspect, provided herein is a pharmaceutical composition comprising highly pure cinacalcet or a pharmaceutically acceptable salt thereof substantially free of at least one, or more, specifically all, of the tetrahydro cinacalcet impurity, cinacalcet N-oxide impurity, cinacalcet benzylamine impurity, and ‘0.66 RRt’ impurity made by the process disclosed herein, and one or more pharmaceutically acceptable excipients.

In still further aspect, encompassed is a process for preparing a pharmaceutical formulation comprising combining highly pure cinacalcet or a pharmaceutically acceptable salt thereof substantially free of at least one, or more, specifically all, of the tetrahydro cinacalcet impurity, cinacalcet N-oxide impurity, cinacalcet benzylamine impurity, and ‘0.66 RRt’ impurity with one or more pharmaceutically acceptable excipients.

In another aspect, the highly pure cinacalcet or a pharmaceutically acceptable salt thereof substantially free of at least one, or more, specifically all, of the tetrahydro cinacalcet impurity, cinacalcet N-oxide impurity, cinacalcet benzylamine impurity, and ‘0.66 RRt’ impurity disclosed herein for use in the pharmaceutical compositions has a 90 volume-percent of the particles (D₉₀) of less than or equal to about 400 microns, specifically less than or equal to about 300 microns, more specifically less than or equal to about 100 microns, still more specifically less than or equal to about 60 microns, and most specifically less than or equal to about 15 microns.

### DETAILED DESCRIPTION

According to one aspect, there is provided a tetrahydro cinacalcet, (R)-α-methyl-N-[3-[3-(trifluoromethyl)phenyl]propyl]-1-(5,6,7,8-tetrahydronaphthalene)methaneamine, having the following structural formula A:

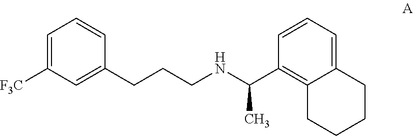

or a pharmaceutically acceptable acid addition salt thereof.

The acid addition salts of tetrahydro cinacalcet can be derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, benzenesulfonic acid, toluenesulfonic acid, citric acid, glutaric acid, citraconic acid, glutaconic acid, and tartaric acid.

Specific pharmaceutically acceptable acid addition salts of tetrahydro cinacalcet are hydrochloride, hydrobromide, oxalate, maleate, fumarate, besylate, tosylate, tartrate, di-p-toluoyl-L-(+)-tartarate, and more specifically tetrahydro cinacalcet hydrochloride.

According to another aspect, there is provided an impurity of cinacalcet, the tetrahydro cinacalcet impurity, (R)-α-methyl-N-[3-[3-(trifluoromethyl)phenyl]propyl]-1-(5,6,7,8-tetrahydronaphthalene)methane amine, of formula A.

The tetrahydro cinacalcet impurity has been identified, isolated and synthesized. The tetrahydro cinacalcet impurity was detected and resolved from cinacalcet by HPLC with an RRt of 1.1. The structure of the compound of formula A was deduced with the aid of ¹H, ¹³C NMR and IR spectroscopy and FAB mass spectrometry. The parent ion at 361 is consistent with the assigned structure.

The tetrahydro cinacalcet disclosed herein is characterized by data selected from a ¹H NMR (500 MHz, CDCl₃) δ (ppm): 1.27 (d, 3H), 1.6-1.8 (m, 6H), 2.4-2.8(m, 8H), 4.0-4.1(m, 1H), 6.9(d, 1H), 7.1-7.2 (m, 1H), 7.2-7.4 (m, 5H); and MS:m/z:361.

The present inventors have found that the tetrahydro cinacalcet impurity is formed as an impurity in the synthesis of cinacalcet due to over reduction of N-BOC protected unsaturated cinacalcet during catalytic hydrogenation process by using hydrogen gas in the presence of hydrogenation catalyst such as palladium hydroxide, for example, as per the process exemplified in the Example 2 as disclosed herein.

Based on the extensive research and experimentation carried out by the present inventors, it has been surprisingly and unexpectedly found that the formation of tetrahydro cinacalcet impurity in the synthesis of cinacalcet or a pharmaceutically acceptable salt thereof can be controlled or substantially removed by using a suitable hydrogen transfer reagent such as formic acid or salts of formic acid such as ammonium formate in the presence of a suitable hydrogenation catalyst in a suitable solvent under appropriate reaction conditions.

According to another aspect, there is provided an isolated tetrahydro cinacalcet impurity. Tetrahydro cinacalcet formed during the synthesis of cinacalcet or a pharmaceutically acceptable salt thereof can be isolated by subjecting the cinacalcet or a pharmaceutically acceptable salt thereof that contains the tetrahydro cinacalcet to column chromatography. The column chromatography comprises using a silica gel, as a stationary phase, and a gradient of eluents that remove tetrahydro cinacalcet from the column on which it adsorbed.

In one embodiment, the tetrahydro cinacalcet of formula A is prepared as per the process exemplified in the Example 14 as disclosed herein.

According to another aspect, there is provided a cinacalcet N-oxide compound, (R)-α-Methyl-N-[3-[3-(trifluoromethyl)phenyl]propyl]-1-naphthalenemethaneamine-N-oxide, having the following structural formula B:

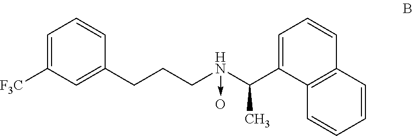

According to another aspect, there is provided an impurity of cinacalcet, cinacalcet N-oxide impurity, (R)-α-Methyl-N-[3-[3-(trifluoromethyl)phenyl]propyl]-1-naphthalene methaneamine-N-oxide, of formula B.

The cinacalcet N-oxide impurity has been identified, isolated and synthesized. The cinacalcet N-oxide impurity was detected and resolved from cinacalcet by HPLC with an RRt of 2.44. The structure of the compound of formula B was deduced with the aid of ¹H, ¹³C NMR and IR spectroscopy and FAB mass spectrometry. The parent ion at 373 is consistent with the assigned structure.

The cinacalcet N-oxide impurity (Formula B) disclosed herein is characterized by data selected from ¹H NMR (500 MHz, CDCl3) δ (ppm): 1.54 (d, 3H), 1.95 (m, 2H), 2.6-2.74 (m, 4H), 4.5 (q, 1H), 4.8 (s, 1H), 7.2-7.6 (m, 8H), 7.7 (m, 1H), 7.8 (m, 1H), 8.2 (d, 1H), and MS:m/s:373.

According to another aspect, there is provided an isolated cinacalcet N-oxide impurity.

In one embodiment, the cinacalcet N-oxide compound of formula B is prepared as per the process exemplified in the Example 13 as disclosed herein.

According to another aspect, there is provided a benzylamine compound, (R)-α-methyl-N-[3-[3-(trifluoromethyl)phenyl]methyl]-1-naphthalenemethaneamine, having the following structural formula C:

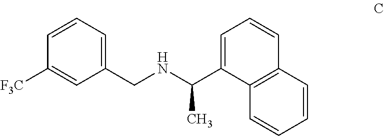

According to another aspect, there is provided an impurity of cinacalcet, benzylamine impurity, (R)-α-methyl-N-[3-[3-(trifluoromethyl)phenyl]methyl]-1-naphthalenemethaneamine, of formula C.

The cinacalcet benzylamine impurity has been identified, isolated and synthesized. The cinacalcet benzylamine impurity was detected and resolved from cinacalcet by HPLC with an RRt of 1.79. The structure of the compound of formula C was deduced with the aid of ¹H, ¹³C NMR and IR spectroscopy and FAB mass spectrometry. The parent ion at 329 is consistent with the assigned structure.

The cinacalcet benzylamine impurity (Formula C) disclosed herein is characterized by data selected from ¹H NMR (500 MHz, CDCl3) δ (ppm): 1.95 (d, 3H), 3.65 (m, 1H), 4.08 (m, 1H), 5.01 (m, 1H), 7.2-7.6 (m,7H), 7.7 (m, 1H), 7.8 (d, 1H), 7.93 (d, 1H), 8.4 (d, 1H), 10.6 (s, 1H), 11.1(s, 1H) and MS:m/s:329.

According to another aspect, there is provided an isolated cinacalcet benzylamine impurity.

In one embodiment, the cinacalcet benzylamine compound of formula C is prepared as per the process exemplified in the Example 12 as disclosed herein.

The present inventors have surprisingly found that the benzylamine impurity is formed as an impurity in the synthesis of cinacalcet due to the contamination of the key starting material 3-trifluoromethylcinnamaldehyde with 3-trifluoromethylbenzaldehyde. The benzylamine (formula C) impurity is formed in the synthesis of cinacalcet during the preparation of unsaturated cinacalcet base by condensation of 3-trifluoromethylcinnamaldehyde (contaminated with 3-trifluoromethylbenzaldehyde) with (R)-(+)-1-(1-naphthyl)ethyl amine in methanol followed by the reaction with sodium borohydride, for example, as per the process exemplified in the Example 4 as disclosed herein. The cinacalcet N-oxide (formula B) impurity is formed in the synthesis of cinacalcet during the catalytic hydrogenation of crude unsaturated cinacalcet or a pharmaceutically acceptable salt in the presence of a suitable hydrogenation catalyst, preferably palladium hydroxide, in a suitable solvent, for example, as per the process exemplified in the Example 9 as disclosed herein.

In addition to the above three impurities, there is another impurity identified at 0.66±0.01 RRt (hereinafter referred to as the ‘0.66 RRt’ impurity or as the ‘single maximum unknown impurity’), whose presence was observed in cinacalcet.

The ‘066 RRt’ impurity disclosed herein is characterized by data selected from ¹H NMR (500 MHz, CDCl₃) δ (ppm): 1.65 (d, 3H), 2.0 2.1 (m, 2H), 2.9-2.98(m, 2H), 4.8-4.9(m, 1H), 5.03-5.07(m, 1H), 7.3-7.6 (m, 8H), 7.7 (m, 1H), 7.9 (m, 1H), 8.1(m, 1H); and MS:m/z:373.

Regarding the specific RRt values of impurities disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.01.

Thus there is a need for a method for determining the level of impurities in cinacalcet samples and removing the impurities.

Extensive experimentation was carried out by the present inventors to reduce the level of the tetrahydro cinacalcet, cinacalcet N-oxide, cinacalcet benzylamine, and ‘0.66 RRt’ impurities in cinacalcet. As a result, it has been found that the tetrahydro cinacalcet, cinacalcet N-oxide, cinacalcet benzylamine, and ‘0.66 RRt’ impurities formed in the preparation of the cinacalcet can be reduced or completely removed by the purification process disclosed herein.

According to another aspect, there is provided a highly pure cinacalcet or a pharmaceutically acceptable salt thereof substantially free of tetrahydro cinacalcet impurity.

In one embodiment, the highly pure cinacalcet or a pharmaceutically acceptable salt thereof disclosed herein is substantially free from at least one, or more, specifically all, of the tetrahydro cinacalcet impurity, cinacalcet N-oxide impurity, cinacalcet benzylamine impurity, and ‘0.66 RRt’ impurity.

According to another aspect, there is provided a highly pure cinacalcet or a pharmaceutically acceptable salt thereof substantially free of at least one, or more, specifically all, of the tetrahydro cinacalcet impurity, cinacalcet N-oxide impurity, cinacalcet benzylamine impurity, and ‘0.66 RRt’ impurity.

As used herein, “highly pure cinacalcet or a pharmaceutically acceptable salt thereof substantially free of tetrahydro cinacalcet impurity” refers to cinacalcet or a pharmaceutically acceptable salt thereof comprising the tetrahydro cinacalcet impurity in an amount of less than about 0.2 area-% as measured by HPLC. Specifically, the cinacalcet, as disclosed herein, contains less than about 0.1 area-%, more specifically less than about 0.05 area-%, still more specifically less than about 0.02 area-% of the tetrahydro cinacalcet impurity, and most specifically is essentially free of the tetrahydro cinacalcet impurity.

In one embodiment, the highly pure cinacalcet or a pharmaceutically acceptable salt thereof disclosed herein comprises the tetrahydro cinacalcet impurity in an amount of about 0.01 area-% to about 0.15 area-%, specifically in an amount of about 0.01 area-% to about 0.05 area-%, as measured by HPLC.

As used herein, “highly pure cinacalcet or a pharmaceutically acceptable salt thereof substantially free of at least one, or more, of the tetrahydro cinacalcet, cinacalcet N-oxide, cinacalcet benzylamine, and ‘0.66 RRt’ impurities” refers to cinacalcet or a pharmaceutically acceptable salt thereof comprising one, or more, of the tetrahydro cinacalcet, cinacalcet N-oxide, cinacalcet benzylamine, and ‘0.66 RRt’ impurities, each one, in an amount of less than about 0.2 area-% as measured by HPLC. Specifically, the cinacalcet, as disclosed herein, contains less than about 0.1 area-%, more specifically less than about 0.05 area-%, still more specifically less than about 0.02 area-% of one, or more, of the tetrahydro cinacalcet, cinacalcet N-oxide, cinacalcet benzylamine, and ‘0.66 RRt’ impurities, and most specifically is essentially free of one, or more, of the tetrahydro cinacalcet, cinacalcet N-oxide, cinacalcet benzylamine, and ‘0.66 RRt’ impurities.

In one embodiment, the highly pure cinacalcet or a pharmaceutically acceptable salt thereof disclosed herein comprises one, or more, of the tetrahydro cinacalcet, cinacalcet N-oxide, cinacalcet benzylamine, and ‘0.66 RRt’ impurities each in an amount of about 0.01 area-% to about 0.15 area-%, specifically in an amount of about 0.01 area-% to about 0.05 area-%, as measured by HPLC.

In another embodiment, the highly pure cinacalcet or a pharmaceutically acceptable salt thereof disclosed herein has a total purity of greater than about 99%, specifically greater than about 99.5%, more specifically greater than about 99.9%, and most specifically greater than about 99.95% as measured by HPLC. For example, the purity of the highly pure cinacalcet or a pharmaceutically acceptable salt thereof is about 99% to about 99.9%, or about 99.5% to about 99.99%.

In another embodiment, the highly pure cinacalcet or a pharmaceutically acceptable salt thereof disclosed herein is essentially free of one, or more, of the tetrahydro cinacalcet, cinacalcet N-oxide, cinacalcet benzylamine, and ‘0.66 RRt’ impurities.

In yet another embodiment, the highly pure cinacalcet or a pharmaceutically acceptable salt thereof disclosed herein is essentially free of the tetrahydro cinacalcet impurity.

The term “cinacalcet or a pharmaceutically acceptable salt thereof essentially free of at least one, or more, of the tetrahydro cinacalcet, cinacalcet N-oxide, cinacalcet benzylamine, and ‘0.66 RRt’ impurities” refers to cinacalcet or a pharmaceutically acceptable salt thereof contains a non-detectable amount of one, or more, of the tetrahydro cinacalcet, cinacalcet N-oxide, cinacalcet benzylamine, and ‘0.66 RRt’ impurities as measured by HPLC.

The term “cinacalcet or a pharmaceutically acceptable salt thereof essentially free of tetrahydro cinacalcet impurity” refers to cinacalcet or a pharmaceutically acceptable salt thereof contains a non-detectable amount of the tetrahydro cinacalcet impurity.

According to another aspect, there is provided a process for preparing highly pure cinacalcet of formula II:

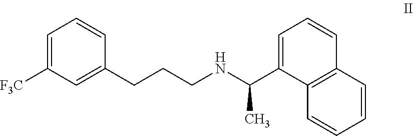

or a pharmaceutically acceptable salt thereof substantially free of tetrahydro cinacalcet impurity, comprising:
a) neutralizing (R)-α-methyl-N-[3-[3-(trifluoromethyl)phenyl]propylene]-1-naphthalene methaneamine hydrochloride salt (unsaturated cinacalcet hydrochloride) of formula III:

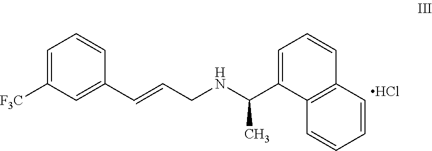

with a first base in a first solvent to provide (R)-α-methyl-N-[3-[3-(trifluoromethyl)phenyl]propylene]-1-naphthalenemethaneamine (unsaturated cinacalcet base) of formula V:

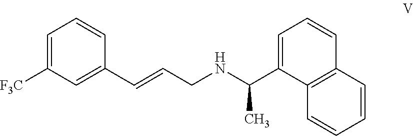

b) reacting the unsaturated cinacalcet base of formula V with a nitrogen protecting agent, optionally in the presence of a second base, in a second solvent to provide N-protected unsaturated compound of formula VI:

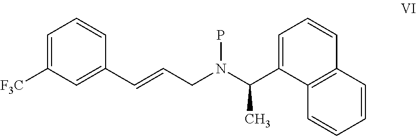

wherein ‘P’ represents a nitrogen protecting group;
c) hydrogenating the compound of formula VI with a hydrogen transfer reagent in the presence of a hydrogenation catalyst in a third solvent to provide the N-protected cinacalcet of formula IV:

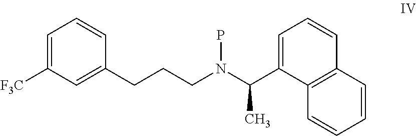

wherein P is as defined in formula VI; and
d) reacting the compound of formula IV obtained in step-(c) with an acid and/or a third base in a fourth solvent to provide highly pure cinacalcet or a pharmaceutically acceptable salt thereof substantially free of the tetrahydro cinacalcet impurity.

In one embodiment, the highly pure cinacalcet or a pharmaceutically acceptable salt thereof obtained by the process disclosed herein is substantially free from at least one, or more, specifically all, of the tetrahydro cinacalcet impurity, cinacalcet N-oxide impurity, cinacalcet benzylamine impurity, and ‘0.66 RRt’ impurity.

Exemplary pharmaceutically acceptable salts of cinacalcet include, but are not limited to, hydrochloride, hydrobromide, oxalate, maleate, fumarate, besylate, tosylate, tartrate, di-p-toluoyl-L-(+)-tartarate. A specific pharmaceutically acceptable salt of cinacalcet is cinacalcet hydrochloride.

Exemplary first solvents used in step-(a) include, but are not limited to, water, an alcohol, a ketone, an ester, acetonitrile, tetrahydrofuran, dimethylformamide, dimethylsulfoxide, dioxane, diethyl carbonate, and mixtures thereof. The term solvent also includes mixtures of solvents.

Specifically, the first solvent is selected from the group consisting of water, methanol, ethanol, isopropyl alcohol, propanol, t-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; more specifically, the first solvent is selected from the group consisting of water, methanol, ethanol, isopropyl alcohol, ethyl acetate, tetrahydrofuran, dimethylformamide, dimethylsulfoxide, and mixtures thereof; and most specifically, the first solvent is selected from the group consisting of water, methanol, ethanol, isopropyl alcohol, ethyl acetate, and mixtures thereof.

In one embodiment, the base used in any of the above steps-(a), (b) and (d) is an organic or inorganic base. Exemplary organic bases are triethylamine, tributylamine, diisopropylethylamine, diethylamine, tert-butylamine, N-methylmorpholine, pyridine, 4-(N,N-dimethylamino)pyridine, and mixtures thereof. Exemplary inorganic bases include, but are not limited to, hydroxides, carbonates and bicarbonates of alkali or alkaline earth metals.

Specific inorganic bases are sodium hydroxide, calcium hydroxide, magnesium hydroxide, potassium hydroxide, lithium hydroxide, sodium carbonate, potassium carbonate, lithium carbonate, sodium bicarbonate, potassium bicarbonate, and more specifically sodium bicarbonate, sodium hydroxide, potassium hydroxide, sodium carbonate, potassium carbonate, and mixtures thereof.

The reaction mass containing the compound of formula V obtained in step-(a) may be subjected to usual work up such as a washing, a filtration, an extraction, an evaporation, or a combination thereof, followed by isolation as solid from a suitable solvent by methods such as cooling, seeding, partial removal of the solvent from the solution, by adding an anti-solvent to the solution, evaporation, vacuum drying, spray drying, freeze drying, or a combination thereof. The reaction mass may be used directly in the next step to produce N-protected unsaturated compound of formula VI, or the compound of formula V may be isolated and then used in the next step.

Exemplary second solvents used in step-(b) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the second solvent is selected from the group consisting of water, methanol, ethanol, isopropyl alcohol, ethyl acetate, tetrahydrofuran, dimethylformamide, dimethylsulfoxide, and mixtures thereof; and most specifically, the second solvent is selected from the group consisting of water, methanol, tetrahydrofuran, and mixtures thereof.

Exemplary nitrogen protecting agents are conventionally used in peptide chemistry and are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981.

In one embodiment, the nitrogen protecting agent is an amine protecting agent selected from the group consisting of an acid anhydride, a mixed anhydride, an acid chloride, an alkyl halide, an aralkyl halide and a silyl compound. A specific nitrogen protecting agent is di-tert-butyl-dicarbonate.

In another embodiment, the nitrogen protecting agent is used in the molar ratio of about 1 to 5 moles, specifically about 1 to 2 moles, per 1 mole of the (R)-α-methyl-N-[3-[3-(trifluoromethyl)phenyl]propylene]-1-naphthalene methaneamine of formula V in order to ensure a proper course of the reaction.

In one embodiment, the reaction in step-(b) is carried out at a temperature of below the boiling temperature of the solvent used, specifically at a temperature of about 0° C. to about 60° C. for at least 1 hour, and more specifically at about 10° C. to about 40° C. for about 5 hours to about 15 hours. In another embodiment, the reaction mass may be quenched with water after completion of the reaction.

Exemplary nitrogen protecting groups ‘P’ include, but are not limited to, acetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl and pivaloyl. Specific nitrogen protecting groups are acetyl, benzyloxycarbonyl (Cbz), trimethylsilyl, triethylsilyl (TES), trimethylsilyethoxymethyl (SEM), tert-butoxycarbonyl (BOC) and pivaloyl. A most specific nitrogen protecting group is tert-butoxycarbonyl (BOC).

The reaction mass containing the N-protected unsaturated compound of formula VI obtained in step-(b) may be subjected to usual work up such as a washing, a filtration, an extraction, an evaporation or a combination thereof. The reaction mass may be used directly in the next step to produce N-protected cinacalcet of formula IV, or the compound of formula VI may be isolated by the methods described hereinabove and then used in the next step.

In one embodiment, a specific N-protected compound of formula IV prepared by the process described herein is N-BOC protected cinacalcet of formula IV(i) (formula IV, wherein P is tert-butoxycarbonyl):

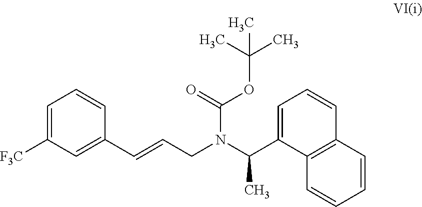

Exemplary hydrogen transfer reagents used in step-(c) include, but are not limited to, formic acid, salts of formic acid such as ammonium formate, sodium formate, trialkyl ammonium formates, hydrazine, 1,3-cyclohexadiene, 1,4-cyclohexadiene and cyclohexene.

As used herein, the term ‘alkyl’ means saturated, acyclic groups which may be straight or branched containing from one to about seven carbon atoms as exemplified by methyl, ethyl, propyl, isopropyl, butyl, hexyl or heptyl.

Specific hydrogen transfer reagents are formic acid, ammonium formate, sodium formate, trimethylammonium formate and tributylammonium formate; and more specifically ammonium formate.

Exemplary hydrogenation catalysts used in step-(c) include, but are not limited to, palladium hydroxide, palladium on carbon, platinum on carbon, platinum oxide, rhodium on carbon, rhodium on alumina, and raney-Ni. A specific hydrogenation catalyst is palladium hydroxide.

Exemplary third solvents used in step-(c) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the third solvent is selected from the group consisting of methanol, ethanol, isopropyl alcohol, n-butanol, and mixtures thereof; and most specifically, the third solvent is methanol.

In one embodiment, the hydrogenation reaction in step-(c) is carried out at a temperature of about 30° C. to the reflux temperature of the solvent used, specifically at a temperature of about 50° C. to the reflux temperature of the solvent used, more specifically at a temperature of about 60° C. to the reflux temperature of the solvent used, and most specifically at the reflux temperature of the solvent used.

The time required for completion of the hydrogenation reaction depends on factors such as solvent used and temperature at which the reaction is carried out.

In another embodiment, the hydrogenation reaction is carried out for at least 30 minutes, specifically for about 1 hour to about 20 hours, and more specifically for about 4 hours to about 8 hours.

For example, if the reaction is carried out in methanol under reflux conditions, for about 5 hours to about 7 hours, is required for the reaction completion.

In one embodiment, the hydrogen transfer reagent is used in the molar ratio of about 0.5 to 5 moles, specifically about 1 to 2 moles, per 1 mole of the compound of formula VI in order to ensure a proper course of the reaction.

In another embodiment, the hydrogenation catalyst is used in the ratio of about 0.05% (w/w) to 10% (w/w), specifically about 0.5% (w/w) to 2.5% (w/w), with respect to the compound of formula VI in order to ensure a proper course of the reaction.

The reaction mass containing N-protected cinacalcet of formula IV obtained in step-(c) may be subjected to usual work up such as a washing, a filtration, an extraction, an evaporation or a combination thereof. The reaction mass may be used directly in the next step to produce substantially pure cinacalcet or a pharmaceutically acceptable salt thereof, or the compound of formula IV may be isolated by the methods described hereinabove and then used in the next step.

Exemplary fourth solvents used in step-(d) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the fourth solvent is selected from the group consisting of water, methanol, ethanol, isopropyl alcohol, n-butanol, and mixtures thereof; and most specifically, the fourth solvent is selected from the group consisting of water, methanol, n-butanol, and mixtures thereof.

If the deprotection reaction in step-(d) is carried out in the presence of a base the product obtained is cinacalcet base, which is in-situ, converted into a pharmaceutically acceptable acid addition salt of cinacalcet using a suitable acid in a suitable solvent. In one embodiment, the pharmaceutically acceptable acid addition salts of cinacalcet can be obtained directly in step-(d) by carrying out the deprotection reaction in the presence of a suitable acid.

Exemplary acids include, but are not limited to, organic and inorganic acids, for example, hydrochloric acid, hydrobromic acid, hydroiodic acid, acetic acid, oxalic acid, fumaric acid, maleic acid, tartaric acid, di-p-toluoyl-L-(+)-tartaric acid, succinic acid, benzenesulfonic acid, toluenesulfonic acid, methanesulfonic acid. Specific acids are hydrochloric acid, oxalic acid and di-p-toluoyl-L-(+)-tartaric acid, and more specifically hydrochloric acid.

The hydrochloric acid used may be in the form of concentrated hydrochloric acid, aqueous hydrochloric acid, in the form of hydrogen chloride gas, or hydrogen chloride dissolved in an organic solvent. The organic solvent used for dissolving hydrogen chloride gas or hydrogen chloride is selected from the group consisting of ethanol, methanol, isopropyl alcohol, ethyl acetate, diethyl ether, dimethyl ether, acetone, and mixtures thereof.

In one embodiment, the reaction in step-(d) is carried out at a temperature of −25° C. to the reflux temperature of the solvent, specifically at a temperature of 0° C. to the reflux temperature of the solvent, more specifically at a temperature of 25° C. to the reflux temperature of the solvent, and most specifically at the reflux temperature of the solvent.

As used herein, “reflux temperature” means the temperature at which the solvent or solvent system refluxes or boils at atmospheric pressure.

The reaction mass containing the pure cinacalcet or a pharmaceutically acceptable salt thereof, preferably cinacalcet hydrochloride, obtained may be subjected to usual work up such as a filtration, a washing, an extractions, an evaporation, or a combination thereof, followed by isolation as a solid from a suitable solvent by the methods described hereinabove.

In one embodiment, the isolation of highly pure cinacalcet or a pharmaceutically acceptable salt thereof substantially free of tetrahydro cinacalcet impurity in step-(d) is carried out by cooling the solution at a temperature of below 30° C. for at least 15 minutes, specifically at about 0° C. to about 30° C. for about 30 minutes to about 20 hours, and more specifically at about 0° C. to about 25° C. for about 1 hour to about 5 hours.

The highly pure cinacalcet or a pharmaceutically acceptable salt thereof substantially free of tetrahydro cinacalcet impurity obtained in step-(d) is recovered by methods such as filtration, filtration under vacuum, decantation, centrifugation, or a combination thereof. In one embodiment, the highly pure cinacalcet or a pharmaceutically acceptable salt thereof substantially free of tetrahydro cinacalcet impurity is recovered by filtration employing a filtration media of, for example, a silica gel or celite.

The highly pure cinacalcet or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

According to another aspect, there is provided a process for the preparation of highly pure cinacalcet or a pharmaceutically acceptable salt thereof substantially free of tetrahydro cinacalcet impurity, comprising:
a) hydrogenating the unsaturated compound of formula VII:

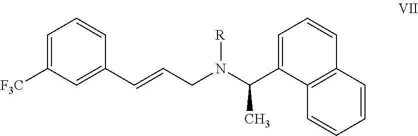

wherein ‘R’ is H or a nitrogen protecting group P; with a hydrogen transfer reagent in the presence of a hydrogenation catalyst in a first solvent to produce a reaction mass containing the saturated compound of formula VIII:

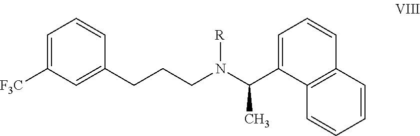

substantially free of tetrahydro cinacalcet impurity, wherein ‘R’ is as defined in formula VII; and
b) optionally, reacting the compound of formula VIII obtained in step-(a) with an acid and/or a base in a second solvent to produce a reaction mass containing the cinacalcet or a pharmaceutically acceptable salt thereof substantially free of tetrahydro cinacalcet impurity; and
c) isolating highly pure cinacalcet or a pharmaceutically acceptable salt thereof substantially free of tetrahydro cinacalcet impurity from the reaction mass obtained in step-(a) or step-(b).

In one embodiment, the nitrogen protecting group ‘P’ is selected from the group as described above. A specific nitrogen protecting group is tert-butoxycarbonyl (BOC).

In another embodiment, the hydrogen transfer reagent used in step-(a) is selected from the group as described above. Specific hydrogen transfer reagents are formic acid, ammonium formate, sodium formate, trimethylammonium formate and tributylammonium formate; and more specifically ammonium formate.

Exemplary hydrogenation catalysts used in step-(a) include, but are not limited to, palladium hydroxide, palladium on carbon, platinum on carbon, platinum oxide, rhodium on carbon, rhodium on alumina, and raney-Ni. A specific hydrogenation catalyst is palladium hydroxide.

Exemplary first solvents used in step-(a) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the first solvent is selected from the group consisting of methanol, ethanol, isopropyl alcohol, n-butanol, and mixtures thereof; and most specifically, the first solvent is methanol.

In one embodiment, the hydrogenation reaction in step-(a) is carried out at a temperature of about 30° C. to the reflux temperature of the solvent used, specifically at a temperature of about 50° C. to the reflux temperature of the solvent used, more specifically at a temperature of about 60° C. to the reflux temperature of the solvent used, and most specifically at the reflux temperature of the solvent used.

The time required for completion of the hydrogenation reaction depends on factors such as solvent used and temperature at which the reaction is carried out.

In another embodiment, the hydrogenation reaction is carried out for at least 30 minutes, specifically for about 1 hour to about 20 hours, and more specifically for about 4 hours to about 8 hours.

For example, if the reaction is carried out in methanol under reflux conditions, for about 5 hours to about 7 hours, is required for the reaction completion.

In one embodiment, the hydrogen transfer reagent is used in the molar ratio of about 0.5 to 5 moles, specifically about 1 to 2 moles, per 1 mole of the compound of formula VII in order to ensure a proper course of the reaction.

In another embodiment, the hydrogenation catalyst is used in the ratio of about 0.05% (w/w) to 10% (w/w), specifically about 0.5% (w/w) to 2.5% (w/w), with respect to the compound of formula VII in order to ensure a proper course of the reaction.

The reaction mass containing saturated compound of formula VIII obtained in step-(a) may be subjected to usual work up such as a filtration, a washing, an extraction, an evaporation or a combination thereof. The reaction mass may be used directly in the next step to produce substantially pure cinacalcet or a pharmaceutically acceptable salt thereof, or the compound of formula VIII may be isolated by the methods described herein and then used in the next step.

Exemplary second solvents used in step-(b) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the second solvent is selected from the group consisting of water, methanol, ethanol, isopropyl alcohol, n-butanol, and mixtures thereof; and most specifically, the second solvent is selected from the group consisting of water, methanol, n-butanol, and mixtures thereof.

In one embodiment, the base used in step-(b) is an organic or inorganic base selected from the group as described above.

If the reaction in step-(b) is carried out in the presence of a base the product obtained is cinacalcet base, which is in-situ, converted into a pharmaceutically acceptable acid addition salt of cinacalcet using a suitable acid in a suitable solvent. In one embodiment, the pharmaceutically acceptable acid addition salts of cinacalcet can be obtained directly in step-(b) by carrying out the deprotection reaction in the presence of a suitable acid.

In one embodiment, the acid is selected from the group as described above. Specific acids are hydrochloric acid, oxalic acid and di-p-toluoyl-L-(+)-tartaric acid.

The hydrochloric acid used may be in the form of concentrated hydrochloric acid, aqueous hydrochloric acid, in the form of hydrogen chloride gas, or hydrogen chloride dissolved in an organic solvent. The organic solvent used for dissolving hydrogen chloride gas or hydrogen chloride is selected from the group as described above.

In one embodiment, the reaction in step-(b) is carried out at a temperature of −25° C. to the reflux temperature of the solvent, specifically at a temperature of 0° C. to the reflux temperature of the solvent, more specifically at a temperature of 25° C. to the reflux temperature of the solvent used, and most specifically at the reflux temperature of the solvent.

The reaction mass containing the pure cinacalcet or a pharmaceutically acceptable salt thereof obtained in step-(b) may be subjected to usual work up such as a filtration, a washing, an extraction, an evaporation or a combination thereof, followed by isolation as solid from a suitable organic solvent by the methods as described hereinabove.

The isolation of highly pure cinacalcet or a pharmaceutically acceptable salt thereof substantially free of tetrahydro cinacalcet impurity in step-(c) is carried out by forcible or spontaneous crystallization.

Spontaneous crystallization refers to crystallization without the help of an external aid such as seeding, cooling etc., and forcible crystallization refers to crystallization with the help of an external aid.

Forcible crystallization is initiated by methods such as cooling, seeding, partial removal of the solvent from the solution, by combining an anti-solvent with the solution or a combination thereof.

In one embodiment, the crystallization is carried out by cooling the solution while stirring at a temperature of below 30° C. for at least 15 minutes, specifically at about 0° C. to about 30° C. for about 30 minutes to about 20 hours, and more specifically at about 0° C. to about 25° C. for about 1 hours to about 5 hours.

The highly pure cinacalcet or a pharmaceutically acceptable salt thereof substantially free of tetrahydro cinacalcet impurity obtained in step-(c) is recovered and further dried by the methods as described hereinabove.

According to another aspect, there is provided a process for the preparation of highly pure cinacalcet or a pharmaceutically acceptable salt thereof substantially free of one, or more, of the cinacalcet N-oxide, cinacalcet benzylamine, and ‘0.66 RRt’ impurities, comprising:
a) reacting crude cinacalcet free base with a nitrogen protecting agent in the presence of a first base in a first solvent to provide N-protected cinacalcet of formula IV:

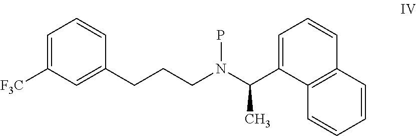

wherein ‘P’ represents a nitrogen protecting group; and
b) converting the compound of formula IV into highly pure cinacalcet or a pharmaceutically acceptable salt thereof substantially free of the impurities by reaction with an acid and/or a second base in a second solvent.

Exemplary first and second solvents used in steps-(a) and (b) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the first and second solvents are, each independently, selected from the group consisting of water, methanol, ethanol, isopropyl alcohol, ethyl acetate, tetrahydrofuran, dimethylformamide, dimethylsulfoxide, and mixtures thereof; and most specifically, selected from the group consisting of water, methanol, tetrahydrofuran, and mixtures thereof.

In one embodiment, the base used in any of the steps-(a) and (b) is an organic or inorganic base selected from the group as described above.

In another embodiment, the nitrogen protecting agent is an amine protecting agent selected from the group as described above. A specific nitrogen protecting agent is di-tert-butyl-dicarbonate.

In another embodiment, the nitrogen protecting agent is used in the molar ratio of about 1.0 to 5 moles, specifically about 1 to 2 moles, per 1 mole of the crude cinacalcet free base in order to ensure a proper course of the reaction.

In one embodiment, the reaction in step-(a) is carried out at a temperature of below the boiling temperature of the solvent used, specifically at a temperature of about 0° C. to about 60° C. for at least 1 hour, and more specifically at a temperature of about 10° C. to about 40° C. for about 5 hours to about 15 hours. In another embodiment, the reaction mass may be quenched with water after completion of the reaction.

In one embodiment, the nitrogen protecting group ‘P’ is selected from the group as described above. A specific nitrogen protecting group is tert-butoxycarbonyl (BOC).

The reaction mass containing the compound of formula IV obtained in step-(a) may be subjected to usual work up by the techniques as described above. The reaction mass may be used directly in the next step to produce substantially pure cinacalcet or a pharmaceutically acceptable salt thereof, or the compound of formula IV may be isolated by the methods as described above and then used in the next step.

If the deprotection reaction in step-(b) is carried out in the presence of a base the product obtained is cinacalcet base, which is in-situ, converted into a pharmaceutically acceptable acid addition salt of cinacalcet using a suitable acid in a suitable solvent. In one embodiment, the pharmaceutically acceptable acid addition salts of cinacalcet can be obtained directly in step-(b) by carrying out the deprotection reaction in the presence of a suitable acid. In another embodiment, the acid is selected from the group as described above. Specific acids are hydrochloric acid, oxalic acid and di-p-toluoyl-L-(+)-tartaric acid.

In one embodiment, the reaction in step-(b) is carried out at a temperature of about −25° C. to the reflux temperature of the solvent, specifically at a temperature of about 0° C. to the reflux temperature of the solvent, more specifically at a temperature of about 25° C. to the reflux temperature of the solvent, and most specifically at the reflux temperature of the solvent.

The reaction mass containing the pure cinacalcet or a pharmaceutically acceptable salt thereof obtained in step-(b) may be subjected to usual work up techniques as described above, followed by isolation as solid from a suitable organic solvent by methods such as cooling, partial removal of the solvent from the solution, addition of precipitating solvent, or a combination thereof.

Crude cinacalcet free base used as starting material can be obtained by the processes disclosed or exemplified hereinafter.

According to another aspect, there is provided a process for the preparation of highly pure cinacalcet or a pharmaceutically acceptable salt thereof substantially free of one, or more, of the cinacalcet N-oxide, cinacalcet benzylamine, and ‘0.66 RRt’ impurities, comprising:
a) neutralizing (R)-α-methyl-N-[3-[3-(trifluoromethyl)phenyl]propylene]-1-naphthalene methaneamine hydrochloride salt (unsaturated cinacalcet hydrochloride) of formula III:

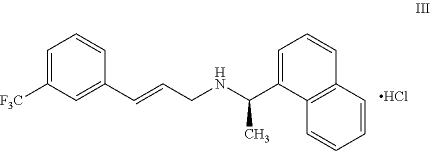

with a first base in a first solvent to provide (R)-α-methyl-N-[3-[3-(trifluoromethyl)phenyl]propylene]-1-naphthalenemethaneamine (unsaturated cinacalcet base) of formula V:

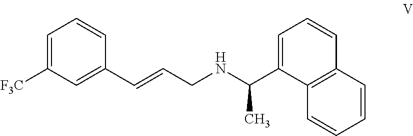

b) reacting the unsaturated cinacalcet base of formula V with a nitrogen protecting agent in the presence of a second base in a second solvent to provide N-protected unsaturated compound of formula VI:

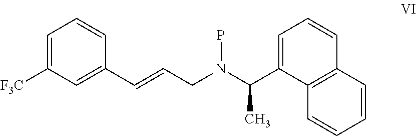

wherein ‘P’ represents a nitrogen protecting group;
c) hydrogenating the compound of formula VI in the presence of a hydrogenation catalyst in a third solvent to provide the N-protected cinacalcet of formula IV:

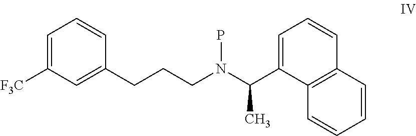

wherein P is as defined in formula VI;
d) converting the compound of formula IV into highly pure cinacalcet or a pharmaceutically acceptable salt thereof substantially free of the impurities by reaction with an acid and/or a third base in a fourth solvent.

Exemplary first, second, third and fourth solvents used in respective steps-(a), (b), (c) and (d) include, but are not limited to, water, methanol, ethanol, isopropyl alcohol, propanol, t-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof.

In one embodiment, the first, second, third and fourth solvents used in the respective steps-(a), (b), (c) and (d) are, each independently, selected from the group consisting of water, methanol, ethanol, isopropyl alcohol, ethyl acetate, tetrahydrofuran, dimethylformamide, dimethylsulfoxide, and mixtures thereof.

In one embodiment, the first, second and third base, used in any of the above steps-(a), (b) and (d), is an organic or inorganic base selected from the group as described above.

Exemplary hydrogenation catalysts used in step-(c) include, but are not limited to, palladium hydroxide, palladium on carbon, platinum on carbon, platinum oxide, rhodium on carbon, and rhodium on alumina. A specific hydrogenation catalyst is palladium hydroxide.

In one embodiment, the hydrogenation reaction in step-(c) is carried out at a temperature of below about 50° C. for at least 30 minutes, specifically at a temperature of about −25° C. to about 40° C. for about 1 hour to about 7 hours, and more specifically at about 0° C. to about 20° C. for about 2 hours to about 5 hours.

In another embodiment, the hydrogenation catalyst is used in the ratio of about 0.05% (w/w) to 10% (w/w), specifically about 0.5% (w/w) to 2.5% (w/w), with respect to the compound of formula VI is used in order to ensure a proper course of the reaction.

In one embodiment, the process steps-(a), (b) and (d) can be carried out by the methods described hereinabove.

According to another aspect, there is provided a highly pure unsaturated cinacalcet or an acid addition salt thereof substantially free of at least one, or both, of the benzylamine impurity and ‘0.66 RRt’ impurity.

As used herein, “highly pure unsaturated cinacalcet or a pharmaceutically acceptable salt thereof substantially free of at least one, or both, of the benzylamine impurity and ‘0.66 RRt’ impurity” refers to unsaturated cinacalcet or a pharmaceutically acceptable salt thereof comprising one, or both, of the benzylamine impurity and ‘0.66 RRt’ impurity, each one, in an amount of less than about 0.2 area-% as measured by HPLC. Specifically, the unsaturated cinacalcet, as disclosed herein, contains less than about 0.1 area-%, more specifically less than about 0.05 area-%, still more specifically less than about 0.02 area-% of one, or both, of the benzylamine impurity and ‘0.66 RRt’ impurity, and most specifically is essentially free of one, or both, of the benzylamine impurity and ‘0.66 RRt’ impurity.

Exemplary acid addition salts of unsaturated cinacalcet base include, but are not limited to, hydrochloride, hydrobromide, sulfate, phosphate, nitrate, tosylate, mesylate, oxalate, p-bromophenylsulfonate, carbonic acid salt, succinate, citrate, benzoate, acetate, maleate, fumarate, tartarate, di-p-toluoyl-tartarate, di-benzoyl-tartarate, di-pivaloyl-tartarate, mandelate, o-chloromandelate, p-chloromandelate, p-bromomandelate and malate. Specific acid addition salts are hydrochloride, oxalate and di-p-toluoyl-L-(+)-tartarate.

According to another aspect, there is provided a process for preparing highly pure unsaturated cinacalcet or a pharmaceutically acceptable salt thereof substantially free of at least one, or both, of the benzylamine impurity and ‘0.66 RRt’ impurity, comprising:
a) contacting crude unsaturated cinacalcet free base with an acid in a first solvent to produce a first reaction mass containing unsaturated cinacalcet acid addition salt;
b) optionally, heating the first reaction mass obtained in step-(a);
c) substantially removing the solvent from the first reaction mass obtained in step-(a) or step-(b) to produce pure unsaturated cinacalcet salt; or
d) isolating pure unsaturated cinacalcet salt from the first reaction mass obtained in step-(a) or step-(b); and/or
e) providing a solution of unsaturated cinacalcet salt obtained in step-(c) or step-(d) in dimethylformamide;
f) combining the solution obtained step-(e) with water to produce a second reaction mass;
g) isolating highly pure unsaturated cinacalcet salt substantially free of the impurities from the second reaction mass obtained in step-(f); and/or
h) neutralizing the pure unsaturated cinacalcet salt, obtained in any of the steps (c), (d) or (g), with a base in a second solvent to provide highly pure unsaturated cinacalcet base substantially free of the impurities.

The acid used in step-(a) is an organic or inorganic acid. In one embodiment, the acid is selected from the group consisting of hydrochloric acid, hydrobromic acid, sulfuric acid, phosphoric acid, nitric acid, p-toluenesulfonic, methanesulfonic acid, oxalic acid, p-bromophenylsulfonic acid, carbonic acid, succinic acid, citric acid, benzoic acid, acetic acid, maleic acid, fumaric acid, tartaric acid, tartaric acid derivatives such as di-p-toluoyl-tartaric acid, di-benzoyl-tartaric acid, di-pivaloyl-tartaric acid; mandelic acid, mandelic acid derivatives such as o-chloromandelic acid, p-chloromandelic acid, p-bromomandelic acid; and malic acid. Specific acids are hydrochloric acid, oxalic acid and di-p-toluoyl-L-(+)-tartaric acid.

In another embodiment, the acid addition salts of unsaturated cinacalcet are hydrochloride, oxalate and di-p-toluoyl-L-(+)-tartarate.

Exemplary first and second solvents used in step-(a) and (h) include, but are not limited to, water, an alcohol, a ketone, an ether, a hydrocarbon, a chlorinated hydrocarbon, a nitrile, an ester, and mixtures thereof.

In one embodiment, the first and second solvents are, each independently, selected from the group consisting of water, methanol, ethanol, propanol, butanol, amyl alcohol, hexanol, acetone, methyl ethyl ketone, methyl isobutyl ketone, methyl tert-butyl ketone, diisopropyl ether, diethyl ether, tetrahydrofuran, dioxane, acetonitrile, n-pentane, n-hexane, n-heptane, cyclohexane, toluene, xylene, methylene chloride, ethyl dichloride, chloroform, carbon tetrachloride, and mixtures thereof; and specifically selected from the group consisting of water, methanol, ethanol, n-butanol, acetonitrile, ethyl acetate, methylene chloride, and mixtures thereof.

In one embodiment, the reaction in step-(a) is carried out at a temperature of about 0° C. to about 100° C., specifically at about 0° C. to about 80° C., and more specifically at about 20° C. to about 60° C.

In another embodiment, the reaction mass in step-(b) is heated at a temperature of about 40° C. to the reflux temperature of the solvent used for at least 20 minutes, and more specifically at the reflux temperature of the solvent used for about 30 minutes to about 5 hours.

The term “substantially removing” the solvent refers to at least 60%, specifically grater than about 85%, more specifically grater than about 90%, still more specifically grater than about 99%, and most specifically essentially complete (100%), removal of the solvent from the solvent solution.

Removal of solvent in step-(c) is accomplished, for example, by substantially complete evaporation of the solvent, concentrating the solution or distillation of solvent, under inert atmosphere.

In one embodiment, the solvent is removed by evaporation. Evaporation can be achieved at sub-zero temperatures by lyophilization or freeze-drying techniques. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The isolation of pure unsaturated cinacalcet salt in step-(d) is carried out by forcible or spontaneous crystallization methods described hereinabove.

In one embodiment, the crystallization is carried out by cooling the solution while stirring at a temperature of below 25° C., specifically at about 0° C. to about 15° C., and still more specifically at about 0° C. to about 5° C.

The pure solid form of unsaturated cinacalcet salt obtained in step-(d) is recovered by the techniques described hereinabove.

Step-(e) of providing a solution of unsaturated cinacalcet salt includes dissolving unsaturated cinacalcet salt in dimethylformamide.

In one embodiment, the unsaturated cinacalcet salt is dissolved in dimethylformamide at a temperature of above about 50° C., specifically at about 65° C. to about 85° C., and more specifically at about 70° C. to about 75° C.

The solution obtained in step-(e) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing unsaturated cinacalcet salt by removing charcoal or silica gel. Preferably, finely powdered carbon is an active carbon. A specific mesh size of silica gel is 40-500 mesh, and more specifically 60-120 mesh.

Combining of the solution with water in step-(f) is done in a suitable order, for example, the solution is added to the water, or alternatively, the water is added to the solution. The addition is, for example, carried out drop wise or in one portion or in more than one portion. The addition is specifically carried out at a temperature of above about 50° C. for at least 15 minutes and more specifically at about 65° C. to about 85° C. for about 20 minutes to about 2 hours. After completion of addition process, the resulting mass is specifically stirred for at least 20 minutes and more specifically for about 30 minutes to about 4 hours at a temperature of about 65° C. to about 85° C.

The isolation of highly pure unsaturated cinacalcet salt obtained in step-(g) is carried out by forcible or spontaneous crystallization methods as described above.

In one embodiment, the crystallization is carried out by cooling the solution while stirring at a temperature of below 25° C., specifically at about 0° C. to about 15° C., and most specifically at about 0° C. to about 5° C.

The highly pure unsaturated cinacalcet salt obtained in step-(g) is recovered by the methods as described above.

In one embodiment, the neutralization reaction in step-(h) is carried out at a temperature of below the boiling temperature of the solvent used, specifically at a temperature of about 0° C. to about 50° C. for at least 30 minutes, and more specifically at a temperature of about 15° C. to about 35° C. from about 2 hours to about 6 hours.

In another embodiment, the neutralization is carried out by adjusting the pH of the reaction mass between about 8 and 14, and specifically between about 9 and 12, with a suitable base.

The base used for neutralization is an organic or inorganic base selected from the group as described above.

The reaction mass containing the unsaturated cinacalcet base obtained step-(h) may be subjected to usual work up techniques as described above, and the highly pure unsaturated cinacalcet base is recovered and further dried by the methods as described above.

The total purity of the unsaturated cinacalcet base or an acid addition salt thereof obtained by the process disclosed herein is of greater than about 98%, specifically greater than about 99%, and more specifically greater than about 99.5% as measured by HPLC.

According to another aspect, there is provided an improved and one pot process for the preparation of cinacalcet or a pharmaceutically acceptable salt thereof, comprising:
a) combining a solution of 3-trifluoromethylcinnamaldehyde in a solvent with (R)-(+)-1-(1-naphthyl)ethyl amine in autoclave vessel to form a first reaction mass;
b) hydrogenating the reaction mass in the presence of a hydrogenation catalyst in the solvent for sufficient time to provide a second reaction mass containing cinacalcet base; and
c) isolating or recovering pure cinacalcet from the second reaction mass containing cinacalcet base and optionally converting the cinacalcet obtained into its pharmaceutically acceptable salts thereof.

Exemplary solvents used in steps-(a) and step-(b) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the solvents are, each independently, selected from the group consisting of water, methanol, ethanol, isopropyl alcohol, n-butanol, and mixtures thereof; and most specifically, selected from the group consisting of water, methanol, n-butanol, and mixtures thereof.

Combining of the solution with (R)-(+)-1-(1-naphthyl)ethyl amine in step-(a) is done in a suitable order, for example, the solution is added to the (R)-(+)-1-(1-naphthyl)ethyl amine, or alternatively, the (R)-(+)-1-(1-naphthyl)ethyl amine is added to the solution. The addition is, for example, carried out drop wise or in one portion or in more than one portion. The addition is specifically carried out at a temperature of below about 50° C. for at least 15 minutes and more specifically at about 15° C. to about 35° C. for about 20 minutes to about 2 hours. After completion of addition process, the resulting mass is specifically stirred for at least 20 minutes and more specifically for about 30 minutes to about 5 hours at a temperature of about 20° C. to about 35° C.

Exemplary hydrogenation catalysts used in step-(b) include, but are not limited to, palladium hydroxide, palladium on carbon, platinum on carbon, platinum oxide, rhodium on carbon, and rhodium on alumina. A specific hydrogenation catalyst is palladium hydroxide.

In one embodiment, the hydrogenation reaction is carried out at a temperature of below about 50° C. for at least 30 minutes, specifically at a temperature of about −25° C. to about 40° C. for about 1 hour to about 7 hours, and more specifically at a temperature of about 0° C. to about 20° C. for about 2 hours to about 5 hours.

In another embodiment, the hydrogenation catalyst is used in the ratio of about 0.05% (w/w) to 10% (w/w), specifically about 0.5% (w/w) to 2.5% (w/w), with respect to the 3-trifluoro methylcinnamaldehyde in order to ensure a proper course of the reaction.

The isolation of pure cinacalcet in step-(c) is carried out by forcible or spontaneous crystallization methods as described above.

In one embodiment, the pure cinacalcet obtained in step-(c) is recovered and further dried by the methods as described above.

Pharmaceutically acceptable salts of cinacalcet can be prepared in high purity by using the substantially pure cinacalcet obtained by the method disclosed herein, by known methods.

According to another aspect, there is provided a process for synthesizing and isolating the tetrahydro cinacalcet of formula A or a pharmaceutically acceptable salt thereof, comprising:
a) hydrogenating cinacalcet base using a Raney Ni catalyst in an alcohol solvent to produce a reaction mass containing crude tetrahydro cinacalcet base;
b) isolating the tetrahydro cinacalcet base from a solvent; and
c) converting the tetrahydro cinacalcet base into a pharmaceutically acceptable salt of tetrahydro cinacalcet, preferably tetrahydro cinacalcet hydrochloride, by reaction with a suitable acid in a solvent.

In one embodiment, the hydrogenation reaction in step-(a) is carried out at a temperature of about 30° C. to the reflux temperature of the solvent, specifically at a temperature of about 50° C. to the reflux temperature of the solvent, more specifically at a temperature of about 60° C. to the reflux temperature of the solvent, and most specifically at the reflux temperature of the solvent.

The time required for completion of the hydrogenation reaction depends on factors such as solvent used and temperature at which the reaction is carried out.

In another embodiment, the hydrogenation reaction in step-(a) is carried out for at least 30 minutes, specifically from about 1 hour to about 25 hours, more specifically from about 5 hours to about 20 hours, and most specifically from about 10 hours to about 18 hours.

For example, if the reaction is carried out in methanol under reflux conditions, from about 14 hours to about 18 hours, is required for the reaction completion.

In one embodiment, the Raney Ni catalyst in the ratio of about 10% (w/w) to 100% (w/w), specifically about 10% (w/w) to 30% (w/w), with respect to the cinacalcet base is used in order to ensure a proper course of the reaction.

Exemplary alcohol solvents used in step-(a) include, but are not limited to, methanol, ethanol, n-propanol, isopropanol, n-butanol, isobutanol, tert-butanol, amyl alcohol, hexanol, and mixtures thereof. Specific alcohol solvents are methanol, ethanol, isopropanol, and mixtures thereof, and more specifically methanol.

The reaction mass containing the tetrahydro cinacalcet base obtained in step-(a) is subjected to usual work up such as a filtration, a washing, an extraction, an evaporations or a combination thereof, and then isolated as a solid from a suitable solvent by conventional methods such as cooling, seeding, partial removal of the solvent from the solution, by adding an anti-solvent to the solution, evaporation, vacuum drying, spray drying, freeze drying, or a combination thereof.

The solvent used for isolating the tetrahydro cinacalcet base in step-(b) is selected from the group consisting of acetone, methanol, ethanol, n-propanol, isopropanol, ethyl acetate, dichloromethane, n-pentane, n-hexane, n-heptane, cyclohexane, toluene, and mixtures thereof, and most specific solvent is n-heptane.

The suitable acids used in step-(c) are selected from the group as described above. Specific acids are hydrochloric acid, oxalic acid and di-p-toluoyl-L-(+)-tartaric acid.

Specific pharmaceutically acceptable acid addition salts of tetrahydro cinacalcet include, but are not limited to, hydrochloride, hydrobromide, oxalate, maleate, fumarate, besylate, tosylate, tartrate, di-p-toluoyl-L-(+)-tartarate, and more specifically tetrahydro cinacalcet hydrochloride.

Further encompassed herein is the use of the highly pure cinacalcet or a pharmaceutically acceptable salt thereof substantially free of at least one, or more, specifically all, of the tetrahydro cinacalcet impurity, cinacalcet N-oxide impurity, cinacalcet benzylamine impurity, and ‘0.66 RRt’ impurity for the manufacture of a pharmaceutical composition together with a pharmaceutically acceptable carrier.

A specific pharmaceutical composition of highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity is selected from a solid dosage form and an oral suspension.

In one embodiment, the highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity has a D₉₀ particle size of less than or equal to about 400 microns, specifically less than or equal to about 300 microns, more specifically less than or equal to about 100 microns, still more specifically less than or equal to about 60 microns, and most specifically less than or equal to about 15 microns.

In another embodiment, the particle sizes of the highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating secondary hyperparathyroidism in patients with chronic kidney disease and hypercalcemia in patients with parathyroid carcinoma, comprising administering a therapeutically effective amount of the highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity, or a pharmaceutical composition that comprises a therapeutically effective amount of highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity, along with pharmaceutically acceptable excipients.

According to another aspect, there is provided pharmaceutical compositions comprising highly pure cinacalcet or a pharmaceutically acceptable salt thereof substantially free of tetrahydro cinacalcet impurity prepared according to the processes disclosed herein and one or more pharmaceutically acceptable excipients.

According to another aspect, there is provided a process for preparing a pharmaceutical formulation comprising combining highly pure cinacalcet or a pharmaceutically acceptable salt thereof substantially free of tetrahydro cinacalcet impurity prepared according to processes disclosed herein, with one or more pharmaceutically acceptable excipients.

According to another aspect, there is provided pharmaceutical compositions comprising highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity prepared according to the processes disclosed herein and one or more pharmaceutically acceptable excipients.

Yet in another embodiment, pharmaceutical compositions comprise at least a therapeutically effective amount of highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure cinacalcet or a pharmaceutically acceptable salt thereof substantially free of at least one, or more, of the tetrahydro cinacalcet impurity, cinacalcet N-oxide impurity, cinacalcet benzylamine impurity, and ‘0.66 RRt’ impurity within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Experimental:

### HPLC Method for Measuring Chemical Purity: Method-I

Chromatographic Parameters:

| | |
|---|---|
| Instrument: | A liquid chromatograph equipped with |
| | UV detector |
| Column: | X-Bridge C18, 150 * 4.6 mm, 3.5μ |
| Column temperature: | 40° C. |
| Wavelength: | 223 nm |
| Flow rate: | 0.8 mL/minute |
| Injection volume: | 10 μL |
| Buffer Preparation: | 1 mL of triethylamine dissolved in 1000 mL of |
| | water, pH: 5.00 adjusted with diluted H₃PO₄, |
| | filtered through 0.45μ membrane filter paper. |
| Mobile Phase-A: | Buffer |
| Mobile Phase-B: | Acetonitrile |
| Diluent: | Water:Acetonitrile (10:90% v/v) |

### HPLC Method for Measuring Chemical Purity: Method-II

Chromatographic Parameters:

| | |
|---|---|
| Instrument: | A liquid chromatograph equipped with UV |
| | detector |
| Column: | X-Bridge C18, 150 * 4.6 mm, 3.5μ |
| Column temperature: | 40° C. |
| Wavelength: | 205 nm |
| Flow rate: | 0.8 mL/minute |
| Injection volume: | 10 μL |
| Buffer Preparation: | 1 mL of triethylamine dissolved in 1000 mL of |
| | water, pH: 5.00 adjusted with diluted H₃PO₄, |
| | filtered through 0.45μ membrane filter paper. |
| Mobile Phase-A: | Buffer |
| Mobile Phase-B: | Acetonitrile |
| Diluent: | Water: Acetonitrile (10:90% v/v) |

The following examples are given for the purpose of illustrating the present disclosure and should not be considered as limitation on the scope or spirit of the disclosure.

### EXAMPLES

### Example 1

### Preparation of (R)-α-Methyl-N-[3-[3-(trifluoromethyl)phenyl]propylene]-1-naphthalene methaneamine hydrochloride (Unsaturated Cinacalcet hydrochloride)

### Step-I: Preparation of Unsaturated Cinacalcet Base

(R)-(+)-1-(1-Naphthyl)ethyl amine (100 g, 0.583 mole) was added to a solution of 3-trifluoromethylcinnamaldehyde (128.6 g, 0.64 mole) in methanol (300 ml) at 25-30° C. for 15 minutes. The reaction mixture was stirred for 4 hours. To the reaction mixture, sodium borohydride (12.14 g, 0.31 mole) was added portion wise slowly at 20-25° C. for about 1 hour. The reaction mixture was stirred at 25-30° C. for 4 hours. The resulting mass was cooled to 5-10° C. Water (600 ml) and ethyl acetate (1000 ml) were added slowly to the reaction mass followed by adjusting pH of the reaction mass to 2-3 with 10% hydrochloric acid. The resulting layers were separated and the organic layer was washed with 20% sodium carbonate solution (400 ml), 20% sodium chloride solution (600 ml) and finally with water (600 ml). The organic layer was concentrated under vacuum at 50° C. to give crude unsaturated cinacalcet base (220 g).

### Step-II: Preparation of Unsaturated Cinacalcet hydrochloride

The crude unsaturated cinacalcet base (220 g, obtained in step-I) was dissolved in acetonitrile (150 ml) followed by the addition of a solution of aqueous hydrochloric acid (73 g) in acetonitrile (150 ml). The precipitated product was stirred at 25-30° C. for 3 hours. The product was filtered, washed with chilled acetonitrile (300 ml) and suck dried for 30 minutes. The wet product was dissolved in dimethylformamide (200 ml) at 70-75° C. and then water (600 ml) was added slowly to the hot solution. The resulting reaction mass was cooled slowly to 0-5° C. The precipitated product was filtered and washed with water (400 ml) and then dried the product at 45-50° C. to yield 100 g of unsaturated cinacalcet hydrochloride (HPLC Purity: 97.9%).

### Example 2

### Preparation of crude (R)-α-Methyl-N-[3-[3-(trifluoromethyl)phenyl]propyl]-1-naphthalenemethaneamine hydrochloride (Crude Cinacalcet hydrochloride)

Unsaturated cinacalcet hydrochloride (10 g, 0.02 moles) was dissolved in methanol (50 ml) followed by the addition of a solution of sodium bicarbonate (4.3 g) in water (50 ml) at 5-10° C. A solution of BOC anhydride (6.6 g, 0.03 moles) dissolved in methanol (50 ml) was added to the above reaction mixture at 5-10° C. for 15 minutes. The reaction mass was maintained at 25-30° C. for 4 hours. After completion of the reaction, ethyl acetate (100 ml) and water (50 ml) were added to the reaction mass and stirred for 15 minutes. The organic layer was separated and washed with water (50 ml) and concentrated under vacuum at 60° C. to produce 12.5 g of N-BOC protected unsaturated cinacalcet. The resulting crude product was dissolved in methanol (100 ml) and hydrogenated with 20% wet palladium hydroxide (0.25 g) under a pressure of 3-4.0 Kg/Cm² for 3 hours at 30-35° C. The catalyst was removed by filtration and evaporated under vacuum at 60° C. to yield 12 g of N-BOC protected cinacalcet freebase. A mixture of concentrated HCl (20 g) and water (30 ml) was added to a solution of the above N-BOC protected cinacalcet freebase dissolved in methanol (80 ml) and refluxed for 3 hours. The reaction mixture was cooled to 0-5° C. and the precipitated product was filtered and washed with a mixture of methanol and water (1:1, 50 ml) followed by water (50 ml). The resulted solid was dried under vacuum at 40-50° C. to yield 8.0 g of crude cinacalcet hydrochloride (Purity by HPLC: 99.33%; content of tetrahydro cinacalcet impurity at 1.1 RRt: 0.57%).

### Example 3

### Preparation of Pure (R)-α-Methyl-N-[3-[3-(trifluoromethyl)phenyl]propyl]-1-naphthalenemethaneamine hydrochloride (Cinacalcet hydrochloride)

Unsaturated cinacalcet hydrochloride (10 g, 0.02 moles) was dissolved in methanol (50 ml) followed by the addition of a solution of sodium bicarbonate (4.3 g) in water (50 ml) at 5-10° C. A solution of BOC anhydride (6.6 g, 0.03 moles) dissolved in methanol (50 ml) was added to the above reaction mixture at 5-10° C. for 15 minutes. The reaction mass was maintained at 25-30° C. for 4 hours. After completion of the reaction, ethyl acetate (100 ml) and water (50 ml) were added to the reaction mass and stirred for 15 minutes. The organic layer was separated and washed with water (50 ml) and concentrated under vacuum at 60° C. to produce 12.5 g of N-BOC protected unsaturated cinacalcet. The resulting crude was dissolved in methanol (100 ml) and added 20% wet palladium hydroxide (0.25 g) and ammonium formate (2.07 g) and the reaction mixture was heated for 6 hours at 60-65° C. The catalyst was removed by filtration and the filtrate was evaporated under vacuum at 50° C. to yield 12 g of N-BOC protected cinacalcet freebase. A mixture of concentrated HCl (20 g) and water (30 ml) was added to a solution of the above N-BOC protected cinacalcet freebase dissolved in methanol (80 ml) and refluxed for 5 hours. The reaction mixture was cooled to 0-5° C. and the precipitated product was filtered and washed with a mixture of methanol and water (1:1, 50 ml) followed by water (50 ml). The resulted solid was dried under vacuum at 40-50° C. to yield 7.5 g of cinacalcet hydrochloride (Purity by HPLC: 99.9%; content of tetrahydro impurity at 1.1 RRt: 0.06%).

### Example 4

### Preparation of (R)-α-Methyl-N-[3-[3-(trifluoromethyl)phenyl]propylene]-1-naphthalene methaneamine hydrochloride (Unsaturated Cinacalcet hydrochloride)

### Step-I: Preparation of Crude Unsaturated Cinacalcet Base

(R)-(+)-1-(1-Naphthyl)ethyl amine (47 g, 1.0 mole) was added to a solution of 3-trifluoromethylcinnamaldehyde (50 g, 1.0 mole) in methanol (250 ml) at 25-30° C. for 15 minutes. The reaction mixture was stirred for 4 hours. To the reaction mixture sodium borohydride (9.45 g, 1.0 mole) was added portion wise slowly at 20-25° C. for about 1 hour. The reaction mixture was stirred at 25-30° C. for 4 hours. The resulting mass was cooled to 5-10° C. Water (100 ml) was added slowly to the reaction mass followed by adjusting pH of the reaction mass to 7.0 with 10% hydrochloric acid. The resulted product was extracted with ethyl acetate (300 ml) and washed thrice with water (200 ml). The organic layer was concentrated under vacuum at 50° C. to give 77.8 g of crude unsaturated cinacalcet base (HPLC Purity: 92.5%).

Content of Impurities: Benzylamine impurity: 1.04%.

### Step-II: Preparation of Unsaturated Cinacalcet hydrochloride

The crude unsaturated cinacalcet base (77.0 g, obtained in step-I) was dissolved in acetonitrile (150 ml) and a solution of aqueous hydrochloric acid (25.2 ml) in acetonitrile (75 ml) was added. The precipitated product was stirred at 25-30° C. for 3 hours. The product was filtered, washed with chilled acetonitrile (150 ml) and then dried at 50-60° C. to give 55 g of unsaturated cinacalcet hydrochloride salt (HPLC Purity: 96.5%).

Content of Impurities: Benzylamine impurity: Not detected.

### Example 5

### Preparation of pure (R)-α-Methyl-N-[3-[3-(trifluoromethyl)phenyl]propylene]-1-naphthalene methaneamine hydrochloride (Unsaturated Cinacalcet hydrochloride)

### Step-I: Preparation of Unsaturated Cinacalcet oxalate Salt

Crude unsaturated cinacalcet free base (5 g, obtained in step-I of example 4) was dissolved in acetonitrile (40 ml) and a solution of oxalic acid (3.9 g, 1.2 mole) in acetonitrile (40 ml) was added at 25-30° C. The stirring was continued for 1-2 hours at 25-30° C. The precipitated salt was filtered and washed with chilled acetonitrile (20 ml). The wet material was dried at 50° C. to give 4.9 g of unsaturated cinacalcet oxalate salt (HPLC Purity: 98.29%).

Content of Impurities: Benzylamine impurity: 0.17%.

### Step-II: Preparation of Unsaturated Cinacalcet Base

Water (250 ml) was added to unsaturated cinacalcet oxalate (25 g, obtained in step-I) under stirring at 25-30° C. followed by addition of 10% sodium hydroxide solution (100 ml) to adjust pH of the reaction mixture up to 10. The reaction mixture was stirred for 1 hour at 25-30° C. followed by the addition of ethyl acetate (250 ml) and then stirred for 30 minutes at 25-30° C. The layers were separated and the aqueous layer was extracted with ethyl acetate (100 ml). The organic layers were combined and washed with brine solution (400 ml). The resulting organic layer was dried over sodium sulfate and evaporated under vacuum at 50° C. to provide 18 g of pure unsaturated cinacalcet base (HPLC Purity: 96.83%).

Content of Impurities: Benzylamine impurity: 0.29%.

### Step-III: Preparation of Pure Unsaturated Cinacalcet hydrochloride

The unsaturated cinacalcet base, obtained in step-II, was dissolved in acetonitrile (90 ml) and concentrated hydrochloric acid (6.3 ml) was added drop wise for 30 minutes at 5-10° C. The reaction mixture was stirred for 3 hours at 25-30° C. The resulting mass was cooled to 0-5° C. and stirred for 1 hour at 0-5° C. The separated solid was filtered, washed with chilled acetonitrile (36 ml) and then dried the product at 50-60° C. to produce 13.0 g of the desired product (Yield: 63%). The obtained product was recrystallized in acetonitrile to afford 11 g of unsaturated cinacalcet hydrochloride (Yield: 85.0%; Purity by HPLC: 98.5%).

Content of Impurities: Benzylamine impurity: 0.02%.

### Example 6

### Preparation of pure (R)-α-Methyl-N-[3-[3-(trifluoromethyl)phenyl]propylene]-1-naphthalene methaneamine hydrochloride (Unsaturated Cinacalcet hydrochloride)

### Step-I: Preparation of Unsaturated Cinacalcet di-p-toluoyl-L-tartaric acid Salt

Crude unsaturated cinacalcet free base (40 g, obtained in example 4) was dissolved in methanol (100 ml) followed by the addition of a solution of di-p-toluoyl-L-tartaric acid (34.2 g) in methanol (100 ml) under stirring at 25-30° C. The stirring was continued for 1-2 hours at 25-30° C. The precipitated product was filtered, washed with methanol (100 ml) and then dried the compound at 50° C. to afford 47.3 g of the unsaturated cinacalcet dip-toluoyl-L-tartaric acid salt (Purity by HPLC: 95.29%).

Content of Impurities: Benzylamine impurity: 0.08%.

### Step-II: Preparation of Unsaturated Cinacalcet Base

Water (450 ml) was added to unsaturated cinacalcet di-p-toluoyl-L-tartrate salt (30 g, obtained in step-I) under stirring at 25-30° C. followed by addition of 10% sodium hydroxide solution (150 ml) to adjust pH of the reaction mixture up to 10. The reaction mixture was stirred for 3 hours at 25-30° C. followed by the addition of ethyl acetate (300 ml) and stirred for 30 minutes at 25-30° C. The layers were separated and the aqueous layer was extracted with ethyl acetate (150 ml). The both organic layers were combined and washed with brine solution (600 ml). The resulting organic layer was dried over sodium sulfate and evaporated under vacuum at 50° C. to get 14.0 g of pure unsaturated cinacalcet free base (Purity by HPLC: 96.42%).

Content of Impurities: Benzylamine impurity: Not detected.

### Step-III: Preparation of Pure Unsaturated Cinacalcet hydrochloride

The unsaturated cinacalcet free base, obtained in step-II, was dissolved in acetonitrile (70 ml) followed by drop wise addition of concentrated hydrochloric acid (5 ml) for 30 minutes at 5-10° C. The reaction mixture was stirred for 3 hours at 25-30° C. The resulting mass was cooled to 0-5° C. and stirred for 1 hour at 0-5° C. The separated solid was filtered, washed with chilled acetonitrile (28 ml) and then dried the product at 50-60° C. to afford the desired product 12.0 g (Yield: 77.0%). The obtained product was recrystallized in acetonitrile to afford 10.2 g of pure unsaturated cinacalcet hydrochloride (Yield: 85.0%; Purity by HPLC: 97.87%).

Content of Impurities: Benzylamine impurity: Not detected.

### Example 7

### Preparation of Pure (R)-α-Methyl-N-[3-[3-(trifluoromethyl)phenyl]propyl]-1-naphthalenemethaneamine hydrochloride (Cinacalcet HCl)

### Step-I: Preparation of Crude Unsaturated Cinacalcet Base

A solution of (R)-(+)-1-(1-Naphthyl)ethyl amine (100 g, 0.583 moles) in methanol (150 ml) was added to a solution of 3-trifluoromethylcinnamaldehyde (128.5 g, 0.642 moles) in methanol (150 ml) at 0-5° C. for 15 minutes. The reaction mixture was stirred for 3 hours. To the reaction mixture, sodium borohydride (12 g, 0.5 moles) was added portion wise slowly at 0-5° C. for about 1 hour. The reaction mixture was stirred at 0-5° C. for 1 hour. Ethyl acetate (600 ml) and water (600 ml) were added to the reaction mixture, stirred for 30 minutes at 25-30° C. followed by adjusting pH of the reaction mass to 2-3.0 with 20% HCl (350 ml) and then stirred for 15 minutes. The resulting organic layer was separated followed by washings with 20% sodium carbonate solution (350 ml) and with brine solution (400 ml). The organic layer was concentrated under vacuum at 50° C. to give 222 g of crude unsaturated cinacalcet base (HPLC Purity: 90.48%).

Content of Impurities: Benzylamine impurity: 1.04%; ‘0.66 RRt’ impurity: 0.23%.

### Step-II: Preparation of Crude Unsaturated Cinacalcet hydrochloride

The unsaturated cinacalcet base (221 g, obtained in step-I) was dissolved in acetonitrile (310 ml) followed by drop wise addition of concentrated hydrochloric acid (86.0 g) for 30 minutes at 5-10° C. The reaction mixture was stirred for 3 hours at 5-10° C. The resulting mass was cooled to 0-5° C. and stirred for 1 hour at 0-5° C. The separated solid was filtered, washed with chilled acetonitrile (200 ml) and then dried the product at 50-60° C. to produce 118.0 g of the desired product (Yield: 54.0%; Purity by HPLC: 97.62%).

Content of Impurities: Benzylamine impurity: 0.02%; ‘0.66 RRt’ impurity: 0.42%.

### Step-III: Purification of Crude Unsaturated Cinacalcet hydrochloride

Unsaturated cinacalcet hydrochloride (25 g; obtained in step-II) was added to dimethylformamide (50 ml) and then heated at 70-75° C. to get a clear solution. This was followed by slow and drop wise addition of water (125 ml) at 70-75° C. for 15 minutes and then stirring for 30 minutes. The reaction mass was initially cooled to 25-30° C. and further cooled to 0-5° C. The precipitated product was filtered, washed with a mixture of chilled dimethylformamide (10 ml) and water (25 ml) and then dried under vacuum at 50-60° C. to give 22.5 g of pure unsaturated cinacalcet hydrochloride (Yield: 90%; Purity by HPLC: 98.01%).

Content of Impurities: Benzylamine impurity: 0.02%; ‘0.66 RRt’ impurity: 0.12%.

### Step-IV: Preparation of Pure cinacalcet hydrochloride

Unsaturated cinacalcet hydrochloride (100 g, 1.0 mole, obtained in step-III) was dissolved in methanol (500 ml) followed by addition of a solution of sodium bicarbonate (42.86 g, 2.0 moles) in water (500 ml) at 5-10° C. A solution of BOC anhydride (66.9 g, 1.2 moles) dissolved in methanol (100 ml) was added to the above reaction mixture at 5-10° C. for 15 minutes. The reaction mass was maintained at 25-30° C. for 4 hours. After completion of the reaction, ethyl acetate (100 ml) and water (1000 ml) were added to the reaction mass and stirred for 15 minutes. The organic layer was separated and washed with water (200 ml) and concentrated under vacuum at 60° C. to produce 125 g of N-BOC protected unsaturated cinacalcet. The resultant crude product was dissolved in methanol (400 ml) and hydrogenated with 20% wet palladium hydroxide (2.5 g) under pressure of 2.0 Kg/Cm² for 3 hours at 5-10° C. The catalyst was removed by filtration and evaporated under vacuum at 60° C. to yield 125 g of N-BOC protected cinacalcet freebase. Concentrated HCl (31.92 g) was added to a solution of the above N-BOC protected cinacalcet freebase dissolved in methanol (800 ml) and refluxed for 3 hours. This was followed by drop wise addition of water (2000 ml) at 25-30° C. for 1 hour. The resulting mass was allowed to cool at 0-5° C. and stirred for 1 hour. The resulting compound was filtered, washed with 50% aqueous methanol (100 ml) and then dried at 60° C. under vacuum to yield 72 g of pure cinacalcet hydrochloride (Purity by HPLC: 99.9%).

Content of Impurities: Benzylamine impurity: 0.02%; N-oxide impurity: Not detected; ‘0.66 RRt’ impurity: 0.01%.

### Example 8

### Preparation of Pure (R)-α-Methyl-N-[3-[3-(trifluoromethyl)phenyl]propyl]-1-naphthalenemethaneamine hydrochloride (Cinacalcet HCl)

### Step-I: Preparation of Crude Cinacalcet Base

Unsaturated cinacalcet hydrochloride (25 g, obtained in step-II of example 4) was dissolved in ethyl acetate (300 ml) at 25-30° C. Water (100 ml) was added to the above solution at 25-30° C. and basified with 25% aqueous sodium carbonate solution (50 ml). The resulting organic layer was separated and taken into an autoclave vessel. 20% wet palladium hydroxide (0.62 g) was added to the above organic layer and hydrogenated at 1.5 Kg/Cm² for 3 hours at 5-10° C. After completion of the reaction, the catalyst was removed by filtration and the solvent was stripped off at 50° C. under vacuum to afford 18 g of cinacalcet base (HPLC purity: 97.61%).

Content of Impurities: Benzylamine impurity: Not detected; N-oxide impurity: 0.24%.

### Step-II: Purification of Crude Cinacalcet Base

Crude cinacalcet free base (18 g, obtained in step-I) was dissolved in methanol (50 ml) and added a solution of sodium bicarbonate (9.8 g) in water (100 ml). BOC anhydride (14.1 g) dissolved in methanol (50 ml) was added to the resultant reaction mixture at 5-10° C. and stirred at 25-30° C. for 3-4 hours. After completion of the reaction, the reaction mass was quenched with water (100 ml) and extracted with ethyl acetate (100 ml). The resulting organic layer was washed twice with water (100 ml) and solvent was evaporated under vacuum below 60° C. to get N-BOC protected cinacalcet base. Concentrated hydrochloric acid (8.5 ml) was added to the solution of N-BOC protected cinacalcet base in methanol (100 ml) and refluxed for 3 hours. Water (200 ml) was added to the reaction mixture drop wise at 60° C. for 1 hour. The reaction mass was allowed to attain to 25-30° C. and stirred 4 hours. The resulting compound was filtered and washed with 50% aqueous methanol (100 ml) and then dried at 60° C. under vacuum to afford 11.2 g of cinacalcet as hydrochloride salt in pure form (Purity by HPLC: 99.89%).

Content of Impurities: Benzylamine impurity: Not detected; N-oxide impurity: Not detected.

### Example 9

### Preparation of pure (R)-α-Methyl-N-[3-[3-(trifluoromethyl)phenyl]propyl]-1-naphthalenemethaneamine hydrochloride (Cinacalcet hydrochloride)

### Step-I: Preparation of Crude Cinacalcet Base

Unsaturated cinacalcet hydrochloride (25 g, obtained in step-II of example 4) was dissolved in ethyl acetate (300 ml). Water (100 ml) was added to the above solution and basified with 25% aqueous sodium carbonate solution (50 ml). The resulting organic layer was separated out and charged into autoclave vessel. 20% wet palladium hydroxide (0.62 g) was added to the above solution and hydrogenated for 3 hours at 5-10° C. under pressure of 1.5 Kg/Cm². After completion of the reaction, the catalyst was removed by filtration and the solvent was stripped off at 50° C. under vacuum to give 21.0 g of crude cinacalcet base (N-oxide impurity: 0.21%; Benzylamine impurity: Not detected).

### Step-II: Purification of Crude Cinacalcet Base

The crude cinacalcet free base (obtained in step-I) was dissolved in tetrahydrofuran (50 ml) followed by the addition of a solution of sodium bicarbonate (9.8 g) in water (100 ml). BOC anhydride (14.1 g) was added to the resultant reaction mixture at 10-15° C. and stirred at 25-30° C. for overnight. After completion of the reaction, the reaction mass was quenched with water (100 ml) and extracted with ethyl acetate (100 ml). The resulting organic layer was washed twice with water (100 ml) and solvent was evaporated under vacuum at below 60° C. to provide N-BOC protected cinacalcet base. Concentrated hydrochloric acid (8.5 ml) was added to the solution of N-BOC protected cinacalcet dissolved in methanol (100 ml) and refluxed for 3 hours. Water (200 ml) was added to the reaction mixture drop wise at 60° C. for 1 hour. The reaction mass was allowed to cool at 25-30° C. and stirred 4 hours. The separated compound was filtered, washed with 50% aqueous methanol (100 ml) and then dried the product under vacuum at 60° C. to afford 19.0 g of the title compound in pure form (Purity by HPLC: 99.8%).

Content of Impurities: Benzylamine impurity: Not detected; N-oxide impurity: Not detected.

### Example 10

### Preparation of pure (R)-α-Methyl-N-[3-[3-(trifluoromethyl)phenyl]propyl]-1-naphthalenemethaneamine hydrochloride (Cinacalcet hydrochloride)

Unsaturated cinacalcet hydrochloride (25 g) was dissolved in ethyl acetate (300 ml). Water (100 ml) was added to the above solution and basified with 25% aqueous sodium carbonate solution (50 ml). This was followed by the evaporation of solvent under vacuum at 50° C. and the crude product was dissolved in tetrahydrofuran (100 ml). Sodium bicarbonate (9.8 g) dissolved in water (100 ml) was added to the above solution followed by the addition of BOC anhydride (14.1 g) at 10-15° C. The reaction mixture maintained for overnight at ambient temperature. After completion of the reaction, water (100 ml) was added to the reaction mass and extracted twice with ethyl acetate (100 ml). The resulting organic layer was washed twice with water (100 ml) and dried over sodium sulfate. The organic layer was concentrated under vacuum at 60° C. to afford 23 g of N-BOC protected unsaturated cinacalcet. The resultant crude product was dissolved in methanol (1000 ml) and hydrogenated with 20% wet palladium hydroxide (0.6 g) under pressure of 1.0 Kg/Cm² for 3 hours at 5-10° C. The catalyst was removed by filtration and evaporated under vacuum at 60° C. to yield N-BOC protected cinacalcet freebase. Concentrated HCl (8.5 ml) was added to a solution of the above crude N-BOC protected cinacalcet free base dissolved in methanol (100 ml) and refluxed for 3 hours. This was followed by drop wise addition of water (200 ml) at 60° C. for 1 hour. The resulting mass was allowed to cool at 25-30° C. and stirred for 4 hours. The resulting product was filtered, washed with 50% aqueous methanol (100 ml) and then dried at 60° C. under vacuum to afford 16.0 g of the title compound (Purity by HPLC: 99.8%).

Content of Impurities: Benzylamine impurity: 0.03%; N-oxide impurity: Not detected.

### Example 11

### Preparation of R)-α-Methyl-N-[3-[3-(trifluoromethyl)phenyl]propyl]-1-naphthalene methaneamine (Cinacalcet Base)

To a solution of 3-trifluoromethylcinnamaldehyde (100 g, 1 mole) in methanol (1300 ml) in an autoclave vessel, (R)-(+)-1-(1-naphthyl)ethyl amine (80.56 g, 1.0 mole) was added drop wise at 5-10° C. The reaction mixture was stirred for 3 hours at 5-10° C. 20% wet palladium hydroxide (5.0 g) was added to the above reaction mixture and hydrogenated at 3.0 Kg/Cm² pressure for 3 hours at 25-30° C. The catalyst was removed by filtration through a celite bed and the resulting solution was concentrated. Water (300 ml) and ethyl acetate (300 ml) was added to obtained crude and acidified with concentrated HCl (25 ml) at 15-20° C. The resulting organic layer was separated and washed thrice with 20% HCl solution (200 ml) followed by basification with 10% sodium carbonate solution (100 ml). The resulting organic layer was washed thrice with brine solution (200 ml) and concentrated on rotavapour under vacuum at 50° C. to yield 123 g of the title compound as oil (Purity by HPLC: 91.0%).

Content of Impurities: Benzylamine impurity: Not detected; N-oxide impurity: Not detected.

### Example 12

### Preparation of (R)-α-Methyl-N-[3-[3-(trifluoromethyl)phenyl]methyl]-1-naphthalenemethane amine (Benzylamine Impurity)

To a mixture of 3-Trifluoromethylbenazaldehyde (2.0 g) in methanol (20.0 ml), (R)-(+)-1-(1-naphthyl)ethyl amine (1.96 g) was added at 25-30° C. for 15 minutes. The reaction mixture was stirred for 4 hours. To the reaction mixture sodium borohydride (1.0 g) was added portion wise slowly at 20-25° C. for about 15 minutes. The reaction mixture was stirred at 25-30° C. for 3 hours. Completion of the reaction was monitored by TLC. Ethyl acetate (50 ml) and water (50 ml) were added to the reaction mass at 25-30° C. 10% HCl solution was added to the reaction mass till pH of the reaction reaches to 7. The resulting organic layer and aqueous layers were separated and the aqueous layer was extracted with ethyl acetate (100 ml). The total organic layer was combined and washed twice with brine solution (100 ml) followed by water (100 ml). The resulting organic layer was dried over sodium sulfate and filtered and then concentrated under vacuum at 50° C. to provide 3.2 g of crude product. The crude product was dissolved in diisopropyl ether (100 ml) and anhydrous HCl was bubbled into the solution up to pH reached 2.0. The precipitated product was stirred for 2 hours at 25-30° C. The resulting product was filtered and washed with diisopropyl ether (50 ml) and then dried at 50-60° C. to afford 3.0 g of the desired product in the form of hydrochloride salt (HPLC Purity: 98.43%).

### Example 13

### Preparation of (R)-α-Methyl-N-[3-[3-(trifluoromethyl)phenyl]propyl]-1-naphthalene methaneamine-N-oxide (N-oxide Impurity)

A mixture of cinacalcet base (15 g), dichloromethane (200.0 mL) and meta-chloro per benzoic acid (17.96 g) was stirred for 3-4 hours at 25-30° C. for reaction completion. 10% sodium bicarbonate solution (100 ml) was added to the reaction mixture at 25-30° C. and stirred for 30 minutes. The resulting organic layer was separated, washed with 10% sodium bicarbonate solution (100 ml) followed by twice with brine solution (200 ml). The resulting organic layer was dried over sodium sulfate and concentrated under reduced pressure at 50° C. to provide the residue. The residue was chromatographed on silica gel eluting with 5% ethyl acetate and hexane mixture to afford 3.7 g of the title compound (Purity by HPLC: 89.99%).

### Example 14

### Preparation of (R)-α-methyl-N-[3-[3-(trifluoromethyl)phenyl]propyl]-1-(5,6,7,8-tetrahydronaphthalene)methane amine hydrochloride (Tetrahydro Cinacalcet hydrochloride)

A solution of cinacalcet base (15 g) in methanol (150 ml) was hydrogenated with raney-Ni catalyst (10 g) at 12-15 kg pressure at 75-80° C. for 16 hours. The catalyst was filtered off and the filtrate was concentrated under vacuum at below 50° C. to get crude product (16 g). The crude product was crystallized from heptane (100 ml) to obtain the free base of tetrahydro cinacalcet. The base was dissolved in acetonitrile (25 ml) and a mixture of concentrated hydrochloric acid (3 g) and water (50 ml) was added at 25-30° C. The reaction mixture was cooled to 0-5° C. and the precipitated product was filtered and washed with water and then dried under vacuum at 45-50° C. to provide 0.75 g of tetrahydro cinacalcet hydrochloride (Purity by HPLC: 96.5%).

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propyl cellulose, hydroxypropylmethylcellulose phthalate, noncrystalline cellulose, magnesium aluminum silicate, triethanolamine, polyvinyl alcohol, and polyvinylpyrrolidone (PVP).

The term “crude cinacalcet or a pharmaceutically acceptable salt thereof” as used herein refers to cinacalcet or a pharmaceutically acceptable salt thereof containing greater than about 0.2 area-%, more specifically greater than about 0.25 area-%, still more specifically greater than about 0.4 area-% and most specifically greater than about 1 area-% of at least one, or more, of the tetrahydro cinacalcet impurity, cinacalcet N-oxide impurity, cinacalcet benzylamine impurity, and ‘0.66 RRt’ impurity.

The term “crude unsaturated cinacalcet or an acid addition salt thereof” as used herein refers to unsaturated cinacalcet or an acid addition salt thereof containing greater than about 0.2 area-%, more specifically greater than about 0.25 area-%, still more specifically greater than about 0.4 area-% and most specifically greater than about 1 area-% of at least one, or both, of the cinacalcet benzylamine impurity and ‘0.66 RRt’ impurity.

As used herein, the term, “detectable” refers to a measurable quantity measured using an HPLC method having a detection limit of 0.01 area-%.

As used herein, in connection with amount of impurities in cinacalcet or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent. “Mean particle size distribution, i.e., (D₅₀)” correspondingly, means the median of said particle size distribution.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Cinacalcet or a pharmaceutically acceptable salt thereof comprising a (R)-α-methyl-N-[3-[3-(trifluoromethyl)phenyl]propyl]-1-[5,6,7,8-tetrahydronaphthalene)methane amine impurity (tetrahydro cinacalcet impurity) in an amount of about 0.01 area-% to about 0.15 area-% as measured by HPLC, wherein the cinacalcet has a purity of about 99% to about 99.99% as measured by HPLC.

2. (canceled)

3. Cinacalcet of claim 1, further comprising one, or more, of a (R)-α-methyl-N-[3-[3-(trifluoromethyl)phenyl]propyl]-1-naphthalenemethaneamine-N-oxide impurity (cinacalcet N-oxide impurity), a (R)-α-methyl-N-[3-[3-(trifluoromethyl)phenyl]methyl]-1-naphthalenemethaneamine impurity (benzyl amine impurity), and a ‘0.66 RRt’ impurity, each, in an amount of less than about 0.2 area-% as measured by HPLC; and wherein the pharmaceutically acceptable salt of cinacalcet is a hydrochloride salt, a hydrobromide salt, an oxalate salt, a maleate salt, a fumarate salt, a besylate salt, a tosylate salt, a tartrate salt or a di-p-toluoyl-L-(+)-tartarate salt.

4. Cinacalcet of claim 3, having a non-detectable amount of one, or more, of the cinacalcet N-oxide, benzylamine, and ‘0.66 RRt’ impurities as measured by HPLC.

5. (canceled)

6. (canceled)

7. (canceled)

8. An isolated tetrahydro cinacalcet, (R)-α-methyl-N-[3-[3-(trifluoromethyl)phenyl]propyl]-1-(5,6,7,8-tetrahydronaphthalene)methaneamine, of formula A: 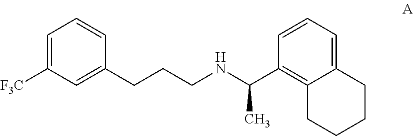
or a pharmaceutically acceptable acid addition salt thereof.

9. An isolated cinacalcet N-oxide compound, (R)-α-Methyl-N-[3-[3-(trifluoromethyl)phenyl]propyl]-1-naphthalenemethaneamine-N-oxide, of formula B:

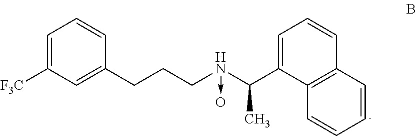

10. An isolated benzylamine compound, (R)-α-methyl-N-[3-[3-(trifluoromethyl)phenyl]methyl]-1-naphthalenemethaneamine, of formula C:

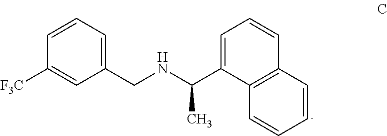

11. A process for preparing the highly pure cinacalcet or a pharmaceutically acceptable salt thereof of claim 1, comprising:
a) neutralizing (R)-α-methyl-N-[3-[3-(trifluoromethyl)phenyl]propylene]-1-naphthalene methaneamine hydrochloride salt (unsaturated cinacalcet hydrochloride) of formula III: 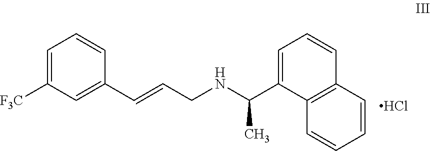
with a first base in a first solvent to provide (R)-α-methyl-N-[3-[3-(trifluoromethyl)phenyl]propylene]-1-naphthalenemethaneamine (unsaturated cinacalcet base) of formula V: 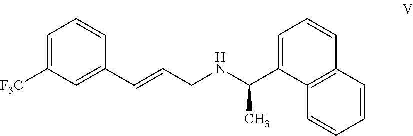
b) reacting the unsaturated cinacalcet base of formula V with a nitrogen protecting agent, optionally in the presence of a second base, in a second solvent to provide N-protected unsaturated compound of formula VI: 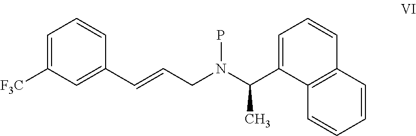
wherein ‘P’ represents a nitrogen protecting group;
c) hydrogenating the compound of formula VI with a hydrogen transfer reagent in the presence of a hydrogenation catalyst in a third solvent to provide the N-protected cinacalcet of formula IV: 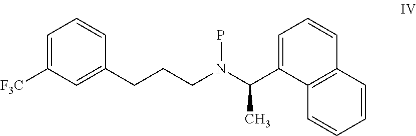
wherein P is as defined in formula VI; and
d) reacting the compound of formula IV obtained in step-(c) with an acid and/or a third base in a fourth solvent to provide highly pure cinacalcet or a pharmaceutically acceptable salt thereof substantially free of the tetrahydro cinacalcet impurity.

12. The process of claim 11, wherein the first, second, third and fourth solvents used in steps-(a), (b), (c) and (d) are, each independently, selected from the group consisting of water, methanol, ethanol, isopropyl alcohol, propanol, t-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; wherein the base used in steps-(a), (b) and step-(b) is, each independently, selected from the group consisting of triethylamine, tributylamine, diisopropylethylamine, diethylamine, tert-butylamine, N-methylmorpholine, pyridine and 4-(N,N-dimethylamino)pyridine, sodium hydroxide, calcium hydroxide, magnesium hydroxide, potassium hydroxide, lithium hydroxide, sodium carbonate, potassium carbonate, lithium carbonate, sodium bicarbonate and potassium bicarbonate; wherein the reaction in step-(b) is carried out at a temperature of below the boiling temperature of the solvent; wherein the hydrogenation reaction in step-(c) is carried out at a temperature of about 30° C. to the reflux temperature of the solvent; and wherein the reaction in step-(d) is carried out at a temperature of −25° C. to the reflux temperature of the solvent.

13. The process of claim 12, wherein the first solvent is selected from the group consisting of water, methanol, ethanol, isopropyl alcohol, ethyl acetate, and mixtures thereof; wherein the second solvent is selected from the group consisting of water, methanol, tetrahydrofuran, and mixtures thereof; wherein the third solvent is selected from the group consisting of methanol, ethanol, isopropyl alcohol, n-butanol, and mixtures thereof; and wherein the fourth solvent is selected from the group consisting of water, methanol, ethanol, isopropyl alcohol, n-butanol, and mixtures thereof.

14. (canceled)

15. The process of claim 11, wherein the nitrogen protecting agent is an amine protecting agent selected from the group consisting of an acid anhydride, a mixed anhydride, an acid chloride, an alkyl halide, an aralkyl halide and a silyl compound; wherein the nitrogen protecting group ‘P’ is selected from the group consisting of acetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl and pivaloyl; wherein the hydrogen transfer reagent used in step-(c) is selected from the group consisting of formic acid, ammonium formate, sodium formate, trialkyl ammonium formates, hydrazine, 1,3-cyclohexadiene, 1,4-cyclohexadiene and cyclohexene; wherein the hydrogenation catalyst used in step-(c) is selected from the group consisting of palladium hydroxide, palladium on carbon, platinum on carbon, platinum oxide, rhodium on carbon, and rhodium on alumina, and raney-Ni; wherein the nitrogen protecting agent is used in a molar ratio of about 1 to 5 moles per 1 mole of (R)-α-methyl-N-[3-[3-(trifluoromethyl)phenyl]propylene]-1-naphthalene methaneamine of formula V; wherein the hydrogen transfer reagent is used in a molar ratio of about 0.5 to 5 moles per 1 mole of the compound of formula VI; and wherein the hydrogenation catalyst is used in a ratio of about 0.5% (w/w) to 10% (w/w) with respect to the compound of formula VI.

16. The process of claim 15, wherein the nitrogen protecting agent is di-tert-butyl-dicarbonate; wherein the nitrogen protecting group ‘P’ is tert-butoxycarbonyl (BOC); wherein the hydrogen transfer reagent is selected from the group consisting of formic acid, ammonium formate, sodium formate, trimethylammonium formate and tributylammonium formate; and wherein the hydrogenation catalyst is palladium hydroxide.

17. (canceled)

18. (canceled)

19. (canceled)

20. (canceled)

21. (canceled)

22. A process for preparing the highly pure cinacalcet or a pharmaceutically acceptable salt thereof of claim 1, comprising:
a) hydrogenating the unsaturated compound of formula VII: 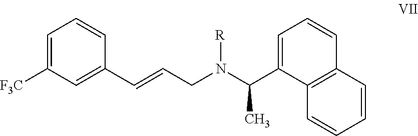
wherein ‘R’ is H or a nitrogen protecting group P; with a hydrogen transfer reagent in the presence of a hydrogenation catalyst in a first solvent to produce a reaction mass containing the saturated compound of formula VIII: 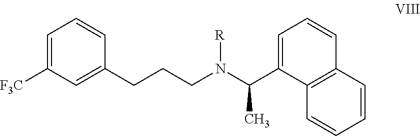
substantially free of tetrahydro cinacalcet impurity, wherein ‘R’ is as defined in formula VII, wherein the hydrogen transfer reagent is selected from the group consisting of formic acid, ammonium formate, sodium formate, trialkyl ammonium formates, hydrazine, 1,3-cyclohexadiene, 1,4-cyclohexadiene and cyclohexene; and wherein the hydrogenation catalyst used in step-(a) is selected from the group consisting of palladium hydroxide, palladium on carbon, platinum on carbon, platinum oxide, rhodium on carbon, and rhodium on alumina, and raney-Ni; and
b) optionally, reacting the compound of formula VIII obtained in step-(a) with an acid and/or a base in a second solvent to produce a reaction mass containing the cinacalcet or a pharmaceutically acceptable salt thereof substantially free of tetrahydro cinacalcet impurity; and
c) isolating highly pure cinacalcet or a pharmaceutically acceptable salt thereof substantially free of tetrahydro cinacalcet impurity from the reaction mass obtained in step-(a) or step-(b).

23. The process of claim 22, wherein the first and second solvents used in steps-(a) and (b) are, each independently, selected from the group consisting of water, an alcohol, a ketone, an ester, acetonitrile, tetrahydrofuran, dimethylformamide, dimethylsulfoxide, dioxane, diethyl carbonate, and mixtures thereof; wherein the nitrogen protecting agent is di-tert-butyl-dicarbonate; wherein the nitrogen protecting group ‘P’ is tert-butoxycarbonyl (BOC); wherein the hydrogen transfer reagent is selected from the group consisting of formic acid, ammonium formate, sodium formate, trimethylammonium formate and tributylammonium formate; and wherein the hydrogenation catalyst is palladium hydroxide.

24. (canceled)

25. (canceled)

26. (canceled)

27. (canceled)

28. (canceled)

29. (canceled)

30. (canceled)

31. (canceled)

32. (canceled)

34. (canceled)

35. A process for preparing highly pure unsaturated cinacalcet or an acid addition salt thereof comprising one, or both, of a (R)-α-methyl-N-[3-[3-(trifluoromethyl)phenyl]methyl]-1-naphthalenemethaneamine (benzyl amine impurity), and a ‘0.66 RRt’ impurity, each, in an amount of less than about 0.2 area-% as measured by HPLC, comprising:
a) contacting crude unsaturated cinacalcet free base with an acid in a first solvent to produce a first reaction mass containing unsaturated cinacalcet acid addition salt;
b) optionally, heating the first reaction mass obtained in step-(a);
c) substantially removing the solvent from the first reaction mass obtained in step-(a) or step-(b) to produce pure unsaturated cinacalcet salt; or
d) isolating pure unsaturated cinacalcet salt from the first reaction mass obtained in step-(a) or step-(b); and/or
e) providing a solution of unsaturated cinacalcet salt obtained in step-(c) or step-(d) in dimethylformamide;
f) combining the solution obtained step-(e) with water to produce a second reaction mass;
g) isolating highly pure unsaturated cinacalcet salt substantially free of the impurities from the second reaction mass obtained in step-(f); and/or
h) neutralizing the pure unsaturated cinacalcet salt, obtained in any of the steps (c), (d) or (g), with a base in a second solvent to provide highly pure unsaturated cinacalcet base substantially free of the impurities.

36. The process of claim 35, wherein the acid used in step-(a) is selected from the group consisting of hydrochloric acid, hydrobromic acid, sulfuric acid, phosphoric acid, nitric acid, p-toluenesulfonic, methanesulfonic acid, oxalic acid, p-bromophenylsulfonic acid, carbonic acid, succinic acid, citric acid, benzoic acid, acetic acid, maleic acid, fumaric acid, tartaric acid, di-p-toluoyl-tartaric acid, di-benzoyl-tartaric acid, di-pivaloyl-tartaric acid, mandelic acid, o-chloromandelic acid, p-chloromandelic acid, p-bromomandelic acid, and malic acid; and wherein the first and second solvents used in step-(a) and (h) are, each independently, selected from the group consisting of water, methanol, ethanol, propanol, butanol, amyl alcohol, hexanol, acetone, methyl ethyl ketone, methyl isobutyl ketone, methyl tert-butyl ketone, diisopropyl ether, diethyl ether, tetrahydrofuran, dioxane, acetonitrile, n-pentane, n-hexane, n-heptane, cyclohexane, toluene, xylene, methylene chloride, ethyl dichloride, chloroform, carbon tetrachloride, and mixtures thereof.

37. (canceled)

38. A one-pot process for the preparation of cinacalcet or a pharmaceutically acceptable salt thereof, comprising:
a) combining a solution of 3-trifluoromethylcinnamaldehyde in a suitable solvent with (R)-(+)-1-(1-naphthyl)ethyl amine in autoclave vessel;
b) hydrogenating the reaction mass in the presence of a hydrogenation catalyst in a solvent for sufficient time to provide a reaction mass containing cinacalcet base; and
c) isolating or recovering pure cinacalcet from the reaction mass and optionally converting the cinacalcet obtained into its pharmaceutically acceptable salts thereof.

39. The process of claim 38, wherein the solvent used in steps-(a) and step-(b) is, each independently, water, an alcohol, a ketone, an ester, acetonitrile, tetrahydrofuran, dimethylformamide, dimethylsulfoxide, dioxane, diethyl carbonate, and mixtures thereof; wherein the hydrogenation catalyst used in step-(b) is selected from the group consisting of palladium hydroxide, palladium on carbon, platinum on carbon, platinum oxide, rhodium on carbon, and rhodium on alumina; and wherein the hydrogenation reaction is carried out at a temperature of below about 50° C. for at least 30 minutes.

40. (canceled)

41. (canceled)

42. (canceled)

43. The highly pure cinacalcet or a pharmaceutically acceptable salt thereof of claim 1, further comprising one or more pharmaceutically acceptable excipients to form a pharmaceutical composition.

44. (canceled)

45. (canceled)

46. (canceled)

47. The pharmaceutical composition of claim 43, wherein the highly pure cinacalcet or a pharmaceutically acceptable salt thereof has a D₉₀ particle size of less than or equal to about 400 microns.

48. (canceled)

49. (canceled)
